(19) United States
(12) Patent Application Publication
(10) Pub. No.: US 2025/0362264 A1
(43) Pub. Date: Nov. 27, 2025

(54) ASSEMBLY AND METHOD FOR MONITORING AN AMMONIA EXHAUST GAS PURIFICATION PLANT

(71) Applicant: Dräger Safety AG & Co. KGaA, Lubeck (DE)

(72) Inventor: Norbert Wruck, Lubeck (DE)

(21) Appl. No.: 19/214,488

(22) Filed: May 21, 2025

(30) Foreign Application Priority Data
May 24, 2024 (DE) 102024114599.4
Aug. 7, 2024 (DE) 102024122491.6

(51) Int. Cl. G01N 27/27; G01N 27/406; G01N 27/407; G01N 27/416; G01N 33/00

(52) U.S. Cl. CPC G01N 27/27; G01N 27/4067; G01N 27/407; G01N 27/4167; G01N 33/0027

(57) ABSTRACT

The present disclosure relates to an assembly and to a method for monitoring an exhaust gas purification plant. The exhaust gas purification plant reduces the content of ammonia in a raw gas and thereby provides a clean gas. A raw gas sensor measures the ammonia content in the raw gas, i.e., upstream of the exhaust gas purification plant. A clean gas sensor measures the ammonia content in the clean gas, i.e., downstream of the exhaust gas purification plant. A signal-processing evaluation unit determines each malfunction period. During a malfunction period, a given quality function is smaller than a specified lower limit. The quality function is the greater, the smaller the ammonia content in the clean gas is at a constant ammonia content in the raw gas.

### CROSS-REFERENCE TO RELATED APPLICATIONS

This application claims the priority of German Patent Application No. 102024114599.4, filed on May 24, 2024; and of German Patent Application No. 102024122491.6, filed on Aug. 7, 2024, each of which is hereby incorporated by reference in its entirety for all nonlimiting purposes.

### SUMMARY

The present disclosure relates to an assembly and to a method which are capable of monitoring at least one exhaust gas purification plant. The or each monitored exhaust gas purification plant is configured to reduce the content of ammonia (NH₃) in a gas.

The term “exhaust gas” generally refers to a gas mixture that is produced in a manufacturing or processing process, wherein the gas mixture contains at least one pollutant. A pollutant is a substance that is or maybe harmful to humans and/or to the environment, and the content (concentration) of the pollutant in an area accessible to humans, in particular in the environment, should therefore be below a specified upper limit. If the gas mixture consists essentially of breathing air, the term “exhaust air” is also used as a special case for exhaust gas.

Such an exhaust gas purification plant is used, for example, in an agricultural enterprise (farm) for animal breeding and in plants for the treatment of sewage sludge or in a plant to produce chemical substances or foodstuffs. Ammonia is often produced in plants such as those plants just mentioned. The exhaust gas purification plant is used to reduce the amount of ammonia escaping into the environment and ideally to completely prevent ammonia from escaping into the environment.

The present disclosure is based on the object of providing a monitoring assembly and a monitoring method capable of monitoring an ammonia exhaust gas purification plant and saving the need for a human to permanently monitor the plant.

The object is achieved by a monitoring assembly having the features described herein and by a monitoring method having the features described herein. Advantageous embodiments are specified in the claims. Advantageous embodiments of the monitoring assembly according to the present disclosure are, as far as appropriate, also advantageous embodiments of the monitoring method according to the present disclosure, and vice versa.

The monitoring assembly according to the present disclosure and the monitoring method according to the present disclosure are capable of monitoring at least one exhaust gas purification plant, optionally multiple exhaust gas purification plants simultaneously or at least in a time-overlapping manner. Several monitored exhaust gas purification plants can be installed at two or more different locations. The or each monitored exhaust gas purification plant is capable of reducing the content of ammonia in a gas mixture—of course only if this gas mixture contains ammonia. The gas mixture which contains, or may at least temporarily contain, the ammonia and which reaches the monitored exhaust gas purification plant, is hereinafter referred to as “raw gas.” The gas mixture, in which the ammonia content has been reduced and which leaves the exhaust gas purification plant, is hereinafter referred to as “clean gas.” Ideally, the clean gas contains no ammonia at all.

The monitoring assembly comprises a first monitoring unit. If the monitoring assembly monitors multiple exhaust gas purification plants simultaneously or at least in a time-overlapping manner, the monitoring assembly comprises one respective monitoring unit for each individual monitored exhaust gas purification plant. The or each monitoring unit is therefore assigned to one respective exhaust gas purification plant monitored.

The monitoring method is carried out using such a monitoring assembly.

The first monitoring unit comprises:
at least one raw gas sensor, optionally multiple raw gas sensors, and
at least one clean gas sensor, optionally multiple clean gas sensors.

The or each optional further monitoring unit also comprises at least one raw gas sensor and at least one clean gas sensor.

The or each raw gas sensor is configured to measure the ammonia content (the ammonia concentration, the ammonia proportion, the ammonia share) in the raw gas. This means: The raw gas sensor is able to measure at least one physical variable that correlates with the ammonia content in the raw gas. Said raw gas reaches the monitored exhaust gas purification plant. The measured variable or the combination of the measured variables together correlates with the ammonia content in the raw gas and is therefore an indicator for the ammonia content in the raw gas.

The terms “raw gas sensor” and “clean gas sensor” may refer to the utilization of an ammonia sensor. A raw gas sensor may be implemented in the same manner than a clean gas sensor.

The or each raw gas sensor is capable of generating a signal, wherein said signal contains information about the ammonia content in the raw gas measured by said sensor.

The or each clean gas sensor is configured to measure the ammonia content in the clean gas. This clean gas leaves the monitored exhaust gas purification plant. This means: The clean gas sensor is able to measure at least one physical variable that correlates with the ammonia content in the clean gas.

The or each clean gas sensor is capable of generating a signal, wherein said signal contains information about the ammonia content in the clean gas measured by said sensor.

Unless otherwise specified, the term “sensor” used below refers to both the or each raw gas sensor and the or each clean gas sensor of the first monitoring unit. Hereinafter, a situation is described in which the monitoring assembly monitors one exhaust gas purification plant and comprises an assigned first monitoring unit. A corresponding modification applies in the embodiment that the monitoring assembly monitors at least two exhaust gas purification plants and comprises a respective assigned monitoring unit for each monitored exhaust gas purification plant.

The monitoring assembly further comprises a signal-processing evaluation unit. The evaluation unit can be implemented as a software program or comprise a software program. A processor of a computer is able to execute the evaluation unit. During execution, the evaluation unit carries out the steps described below. The evaluation unit can also be implemented as a processor or as a signal processing unit or can comprise a processor, wherein the processor is configured to carry out the steps described below.

The evaluation unit is able to determine the ammonia content in the raw gas and the ammonia content in the clean gas of the monitored exhaust gas purification plant. For this purpose, the evaluation unit is able to receive and process messages. The received and processed messages comprise the signals of the sensors of the first monitoring unit.

The evaluation unit is able to determine each malfunction period that occurs within a specified monitoring period. For this purpose, the evaluation unit can use the determined (measured) ammonia content in the raw gas and the determined ammonia content in the clean gas. Alternatively, the evaluation unit is able to determine that there is no malfunction period in the monitoring period.

A malfunction period (error period, faulty period) is a period in which a specified quality function is continuously smaller than a specified lower limit. More precisely, a malfunction period is a period in which every determined functional value of the quality function is smaller than the limit. This quality function is specified in a computer-evaluable form, assumes for several time points the respective functional value, depends on the ammonia content in the raw gas and on the ammonia content in the clean gas and has the following property: A functional value of the quality function is the greater, the smaller the ammonia content in the clean gas is at a constant ammonia content in the raw gas. The functional value of the quality function is therefore the greater, the more ammonia the exhaust gas purification plant removes from the raw gas.

In some examples, a functional value of the quality function is the greater, the smaller the quotient of the ammonia content in the clean gas (numerator) and the ammonia content in the raw gas (denominator) is. In another embodiment, the functional value of the quality function is the greater, the larger the quotient of Δ (numerator) and the ammonia content in the raw gas (denominator) is, with Δ being the difference between the ammonia content in the raw gas and the ammonia content in the clean gas. If the exhaust gas purification plant has not completely failed and the raw gas contains ammonia, then Δ>0 and the denominator>0 holds. This quality function can also be referred to as the relative purification efficiency and is at best (at most) equal to 1.

Preferably, when searching for malfunction periods, only those periods that are at least as long as a specified minimum duration are found (determined). In some cases, this can reduce the influence of outliers (freak values).

The monitoring assembly is preferably configured as follows: The evaluation unit receives a respective sequence of signal values from each sensor of the first monitoring unit, wherein the time interval between two consecutive signal values is not greater than a specified time interval. A period that is longer than said specified time interval and in which no signal value from at least one sensor is received by the evaluation unit is also considered a malfunction period. During said longer period, the sensor and/or the data transmission from the sensor or from the first monitoring unit to the evaluation unit may have failed.

The monitoring method according to the present disclosure is carried out automatically using a monitoring assembly according to the present disclosure and comprises the corresponding steps.

In many cases, legal and regulatory requirements provide that emissions of ammonia into the environment must be limited. Therefore, an exhaust gas purification plant is required for many businesses and plants in which ammonia is at least temporarily produced. Such an exhaust gas purification plant removes at least part of the ammonia from a raw gas produced (e.g., generated) during operation, i.e. the plant reduces the ammonia content. In one application, the business is an agricultural enterprise (e.g., a farm) where livestock is kept and fed. In another application, the business is a plant that processes sewage sludge or a plant that produces chemical substances or foodstuffs.

The present disclosure makes it possible to remotely monitor the exhaust gas purification plant. The sensors and the evaluation unit work automatically, and usually only the sensors need to be checked by a human from time to time.

According to the present disclosure, the first monitoring unit comprises at least one raw gas sensor and at least one clean gas sensor. Preferably, each sensor is arranged spaced apart from the or any other sensor of the first monitoring unit. The or each raw gas sensor measures the ammonia content upstream of the exhaust gas purification plant, and the or each clean gas sensor measures the ammonia content downstream of the same exhaust gas purification plant. Thanks to this feature, it is not necessary to derive the ammonia content downstream of the exhaust gas purification plant from a measured ammonia content upstream of the exhaust gas purification plant, or vice versa. Such derivation would in general require measuring or specifying at least one further variable, in particular a variable of the monitored exhaust gas purification plant itself or of an environmental condition, or to specify and use a default value. Thanks to the present disclosure, it is not necessary to measure such a further variable or to specify a default value. In many cases, the fact that the ammonia content in the raw gas and the ammonia content in the clean gas are measured relatively reliably increases reliability.

Because a raw gas sensor and a clean gas sensor are used, it is possible, but in many cases not necessary, to measure an operating parameter of the monitored exhaust gas purification plant. Examples of such operating parameters are a volume flow achieved by a fluid conveying unit of the exhaust gas purification plant, or a power consumption of the fluid conveying unit, or a consumption of a chemical used for exhaust gas purification, or a volume flow or property of a cleaning liquid. Because it is not necessary to measure an operating parameter, it is also not necessary to adapt the monitoring assembly to a specific transmission protocol or a specific data format of the monitored exhaust gas purification plant. Furthermore, the reliability with which the monitoring assembly monitors the exhaust gas purification plant does not depend on a sensor of the monitored exhaust gas purification plant and its reliability. The feature that no parameter of the monitored exhaust gas purification plant needs to be measured makes it easier in many cases to implement a monitoring assembly according to the present disclosure, even for monitoring an already existing exhaust gas purification plant.

A simple example is provided to illustrate the advantage of the present disclosure. According to said example, a heating plant heats a building. It is to be determined whether the building is sufficiently heated, i.e. whether the heating plant operates properly. It would be possible to measure an operating parameter of the heating plant, for example the consumption of fossil fuel or electrical energy. This is possible, but not necessary, if an indoor thermometer (e.g., corresponding to the clean gas sensor) measures the temperature inside the building and an outdoor thermometer (e.g., corresponding to the raw gas sensor) measures the temperature outside the building.

According to the present disclosure, both the or each raw gas sensor and the or each clean gas sensor measure an ammonia content (the ammonia concentration, the ammonia proportion, the ammonia share) in a gas. It is possible, but thanks to the present disclosure in many cases not necessary, to measure the mass of ammonia in a gas. It is furthermore possible, but thanks to the present disclosure in many cases not necessary, to measure a volume flow (volume flow rate, volume stream) or a mass flow of the ammonia or of the entire gas in order to derive the ammonia content. Often, the concentration of ammonia in a gas can be measured with greater reliability than the mass or mass flow or volume flow. Instead of a mass or mass flow or volume flow, a physical variable that directly correlates with the ammonia content in a gas is measured in many cases.

The determined ammonia content in the clean gas can be used to determine the remaining ammonia emissions. However, in many cases, the ammonia content in the clean gas alone is not sufficient to determine whether the exhaust gas purification plant is still working (operating) properly or not. For example, if the raw gas contains little ammonia, the clean gas will also contain little ammonia, even if the exhaust gas purification plant does not work properly or does not work at all. As an additional example, if the ammonia content in the raw gas is below a specified upper limit, the ammonia content in the clean gas is also below said limit, even if the exhaust gas purification plant is not working at all. If raw gas with a larger volume flow or with a larger ammonia content is later fed into the exhaust gas purification plant, an upper limit for the amount of ammonia emitted can quickly be exceeded. In many cases, the present disclosure makes it possible to detect such an undesirable situation at an early stage or even prevent it from occurring in the first place (at all).

According to the present disclosure, the respective ammonia content is measured both in the clean gas and in the raw gas, i.e., both downstream and upstream of the exhaust gas purification plant. The quality function used according to the present disclosure depends on both the ammonia content in the clean gas and the ammonia content in the raw gas. The quality function is an indicator of how well and to what extent the exhaust gas purification plant reduces the content of ammonia in the raw gas.

According to the present disclosure, the evaluation unit determines each malfunction period in a specified monitoring period. During a malfunction period, the quality function continuously (throughgoing) takes a functional value that is smaller than the specified lower limit. Usually, the current functional value of the quality function varies (fluctuates) both within and outside of a malfunction period. In many cases, the determined malfunction periods can be used to assess whether the exhaust gas purification plant actually worked incorrectly during a malfunction period or whether another influencing factor led to low quality function values, for example little raw gas (in particular low volume flow or mass flow) or little ammonia in the raw gas (low ammonia content).

The quality function used according to the present disclosure depends on the ammonia content in the raw gas and the ammonia content in the clean gas. It does not depend directly on the amount of ammonia in the raw gas or on the amount of ammonia emitted as part of the clean gas. Therefore, the quality function is in many cases a better measure of the quality of the exhaust gas purification plant than the emitted amount of ammonia.

The present disclosure does not require the use of an analytical model or a trained classifier, wherein the model or the trained classifier describes the behavior of the exhaust gas purification plant. Developing and testing such an analytical model takes time. In many cases, such an analytical model also comprises operating parameters of the monitored exhaust gas purification plant as model parameters, which parameters would then have to be measured during operation. A sufficiently large and reliable sample is needed to establish a classifier. Another disadvantage of a classifier may be that a sample from a specific exhaust gas purification plant cannot be used for another exhaust gas purification plant.

In some examples, the first monitoring unit comprises two raw gas sensors, optionally three or even more raw gas sensors. This embodiment makes it possible, in some examples, to measure the ammonia content in the raw gas at two or more different measuring positions and to determine an ammonia content averaged over space. In some examples, this embodiment makes it possible to detect the failure of a raw gas sensor and to continue monitoring the exhaust gas purification plant despite the failed raw gas sensor. This is described in more detail below.

The evaluation unit is able to automatically check whether a specified failure criterion for one of the at least two raw gas sensors of the first monitoring unit is met or not. The failure criterion is met if at least one of the following conditions has occurred:
The absolute or percentage deviation between the ammonia content measured by a first raw gas sensor of the first monitoring unit and the ammonia content measured by the or at least one other (second) raw gas sensor of the first monitoring unit is greater than a specified lower limit. The measured values refer—within a tolerance—to the same time point.
The change in this absolute or percentage deviation is greater than a specified lower limit.
The temporal course of the ammonia content measured by a raw gas sensor falls (decreases) very rapidly, namely, more rapidly than a specified upper limit, to a low value, for example to zero. In reality, a very rapid decrease of the ammonia content usually does not occur.

Such a situation usually does not occur if all raw gas sensors of the first monitoring unit are intact. The specified failure criterion is met, in particular, if a measured ammonia content suddenly falls to zero and preferably remains at zero for a sufficiently long time, but another ammonia content in the raw gas measured by the same monitoring unit does not.

A fault in a raw gas sensor almost always results in this sensor measuring too low an ammonia content in the raw gas. In extreme cases, a failure may even result in the sensor not detecting any ammonia at all, even though ammonia is present in the raw gas. Usually, however, a sensor fault does not result in the faulty sensor measuring too high an ammonia content. If the evaluation unit has detected the event that a failure criterion is met and a raw gas sensor must therefore have failed, the evaluation unit automatically decides which raw gas sensor has failed and which has not. The evaluation unit makes this decision as follows: The sensor, that measures the smaller or smallest or very strongly decreasing ammonia content in the raw gas and provides a corresponding signal, is treated as a failed sensor. The measured value of said sensor is not used. In other words: The evaluation unit uses the greatest ammonia content or, more generally, in the case of n raw gas sensors, the n−1 greatest measured ammonia contents to determine the actual ammonia content. If the failure criterion is not met, the evaluation unit uses the respective signal of each raw gas sensor and aggregates the measured ammonia contents, for example as an arithmetic or weighted mean or as a median.

Accordingly, in some examples, the first monitoring unit comprises at least two clean gas sensors. The embodiment just described for automatically detecting the failure of a raw gas sensor is preferably accordingly used to detect the failure of a clean gas sensor and still measure the ammonia content in the clean gas.

In some examples, the evaluation unit is able to determine an availability rate of the exhaust gas purification plant in the specified monitoring period. For this determination, the evaluation unit uses the determined malfunction periods and the respective duration of each malfunction period, taking into account those malfunction periods that fall within the monitoring period and preferably taking into account only malfunction periods that are at least as long as a specified minimum duration. According to the present disclosure, during a malfunction period, every functional value of the quality function takes a respective value that is smaller than the specified lower limit. Outside a malfunction period, the functional values of the quality function usually take values equal to or greater than the lower limit above. The determined availability rate indicates the time proportion in the monitoring period spent in total on periods in which the quality function was greater than or equal to the lower limit. With other words: The availability rate is the share in time of the malfunction periods. The shorter the malfunction periods for a specified monitoring period are in entirety, the higher is the availability rate. The availability rate is 1 if the exhaust gas purification plant works faultlessly throughout the entire monitoring period, i.e., if no malfunction period is detected. If it works incorrectly during the entire monitoring period, the availability rate is 0.

In some examples, the evaluation unit is capable of generating a graphical representation and causing this graphical representation to be visually output. This graphical representation has a first axis for time and a second axis for the ammonia content in the clean gas. The evaluation unit determined this ammonia content depending on the signals of the sensors of the assigned monitoring unit. Usually, the first axis is the x-axis, and the second axis is the y-axis, with the y-axis preferably being perpendicular to the x-axis. The graphical representation shows the temporal course of the determined ammonia content in the clean gas. The graphical representation further shows each determined malfunction period. In some examples, the graphical representation also shows the temporal course of the determined ammonia content in the raw gas, but this is not required. In the graphical representation, each section of the temporal course of the ammonia content in the clean gas that falls within a malfunction period is highlighted. For example, the area between the section and the first axis (time axis) is highlighted. This representation makes it possible for a viewer to identify the malfunction periods quickly and ergonomically, even on a relatively small screen, for example on a smartphone or tablet, or if several plants are monitored simultaneously. In many cases, the viewer can view this representation on a relatively small screen near the exhaust gas purification plant.

The following embodiment specifies at least one sensor of the first monitoring unit in more detail. It is possible that every sensor of the first monitoring unit is constructed in this way, at least every sensor that is able to measure an ammonia content.

According to this embodiment, the sensor comprises a sensor cell with a measuring chamber. The sensor cell is able to measure the ammonia content in a gas sample, wherein said gas sample is located in the measuring chamber. The sensor further comprises a tubular feed unit. Said feed unit extends along a longitudinal axis. While the monitoring assembly is used, the feed unit is arranged vertically or obliquely below the measuring chamber, and the longitudinal axis is therefore arranged vertically or obliquely in space.

The sensor further comprises a heating element. The heating element is configured to heat the interior of the feed unit. By heating, the heating element is able to cause a convection flow (chimney effect) to be generated in the feed unit. Said convection flow conveys a gas sample from the environment through the feed unit and vertically or obliquely upward into the measuring chamber.

This embodiment eliminates the need to provide a pump or other fluid conveying unit to convey a gas sample into the measuring chamber. The heating element usually consumes less electrical energy compared to a fluid conveying unit. This is advantageous, in particular, if the sensor is not or cannot be connected to a stationary power supply network, at least temporarily, and therefore comprises its own power supply unit. In addition, unlike a fluid conveying unit, the heating element has no moving part. In general, a moving part wears out faster than the heating element and can cause vibrations. In some examples, the generated convection flow conveys a gas sample into the measuring chamber faster than if the gas sample were to enter the measuring chamber solely by diffusion.

In some examples, a tubular protective element is assigned to at least one sensor of the first monitoring unit. Said protective element extends along a longitudinal axis. While the monitoring assembly is used, the longitudinal axis of the protective element is arranged vertically or obliquely. The sensor is arranged inside the protective element. A gas sample flows from one end face of the protective element to the sensor. This embodiment can be combined with the embodiment just described, in which a convection flow conveys a gas sample into the measuring chamber.

The embodiment having the tubular protective element reduces the risk of an airflow (air current) near the sensor distorting a measurement result. The lateral surface of the protective element reduces the influence of the air flow on the process of a gas sample entering the measuring chamber of the sensor. A sufficiently large angle occurs between the air flow and the longitudinal axis of the protective element because, at least outdoors, an air flow usually flows approximately horizontally.

In a preferred embodiment, the first monitoring unit additionally comprises a first communication unit. The monitoring assembly further comprises a stationary or mobile central computer. The central computer is arranged at a distance from the first monitoring unit, preferably also outside a building or other region in which the or an exhaust gas purification plant to be monitored is located. If the monitoring assembly monitors multiple exhaust gas purification plants simultaneously, the central computer is preferably located at a distance from each monitored exhaust gas purification plant. The evaluation unit is part of the central computer.

A respective data connection is established or can be established permanently or at least temporarily between each sensor and the first communication unit. In addition, a data connection is established or can be established permanently or at least temporarily between the first communication unit and the central computer. Said data connection is preferably implemented using radio waves, i.e., wirelessly.

The first communication unit is able to receive and process the respective signal of each sensor. The first communication unit is able to generate for each received signal a respective message and to cause the generated message to be transmitted to the central computer. The message comprises information about the ammonia content measured by the sensor from which the signal originated.

It is possible to remotely monitor multiple (several) exhaust gas purification plants simultaneously using the monitoring assembly according to the present disclosure. In this application, therefore, usually signals of different monitoring units reach the central computer. The embodiment described below makes it easier for the central computer to assign each received signal to the correct (proper) monitoring unit and thus to the correct monitored exhaust gas purification plant. The embodiment described below makes possible, but does not require, a computer-accessible list to be maintained and continuously updated in the central computer, wherein said list identifies all currently monitored exhaust gas purification plants. Rather, a further communication unit can also “log in” to the central computer while the monitoring unit is in operation.

According to this embodiment, the first communication unit of the first monitoring unit comprises a data memory. Or the first communication unit has permanent or at least temporary read access to such a data memory. The following information is stored on the data memory, in some examples:
a unique identifier of the first communication unit, and/or
a respective unique identifier for each sensor.

The unique identifier of the first communication unit distinguishes the first communication unit from any other communication unit that is at least temporarily in data connection with the central computer. The unique identifier of a sensor distinguishes this sensor from any other sensor of the first monitoring unit. It is possible, but not necessary, that the unique identifier of a sensor also distinguishes said sensor from any sensor of any other monitoring unit of the monitoring assembly.

According to the embodiment just described, the first communication unit receives a respective signal from each sensor of the first monitoring unit, wherein said signal has been generated by said sensor and comprises information about the ammonia content measured by said sensor. The first communication unit generates a message, wherein said message comprises information about the measured ammonia content. The first communication unit causes this message to be transmitted to the central computer. According to the embodiment just described, the first communication unit generates the message such that the message additionally comprises the identifier of the first communication unit and the identifier of the sensor that has measured the ammonia content and generated the signal.

Optionally, at least one of the following pieces of information is stored on the data memory of the first monitoring unit:
an identifier of the operating principle by which the exhaust gas purification plant removes ammonia, and/or
an identifier indicating how many raw gas sensors and how many clean gas sensors the first monitoring unit comprises, i.e., identifiers for two numbers.

The identifier of the operating principle comprises in particular an identifier of whether the exhaust gas purification plant works purely chemically, or entirely or at least partially biologically.

According to the embodiment just described, the first communication unit thus generates a message, wherein said message comprises
an identifier of the first monitoring unit,
information about the ammonia content measured by a sensor of the first monitoring unit, and
an identifier of the sensor.

Optionally, the generated message also comprises an identifier indicating how many raw gas sensors and how many clean gas sensors the first monitoring unit comprises and/or an identifier of the applied operating principle of the exhaust gas purification plant. This embodiment eliminates the need to provide a separate evaluation unit on the central computer for each monitored exhaust gas purification plant. Rather, it is usually sufficient to keep multiple variants of the evaluation unit in stock, with each variant relating to a respective number of raw gas sensors and a number of clean gas sensors. In the case of M raw gas sensors and N clean gas sensors, the evaluation program comprises at least M*N different variants. The appropriate variant is selected and used.

Optionally, it also depends on the operating principle of the monitored exhaust gas purification plant which variant the evaluation unit will apply to evaluate the signals of the sensors of the monitoring unit assigned to said exhaust gas purification plant. In the case of K different potential operating principles, there are a total of M*N*K different variants. In one implementation, an identifier indicating the operating principle used by the assigned exhaust gas purification plant is stored on the data memory of the first monitoring unit. This identifier is transmitted to the central computer and evaluated by the evaluation unit. Preferably, the message, which the first monitoring unit generates, and which comprises a signal of a sensor, additionally comprises the identifier of the operating principle actually applied.

A further embodiment is described below. Said further embodiment can be combined with the embodiment in which an identifier of the operating principle applied is transmitted and evaluated.

According to the further embodiment, the raw gas is passed through a cleaning liquid, wherein the cleaning liquid belongs to the monitored exhaust gas purification plant and is preferably an aqueous solution. While the raw gas is passed through the cleaning liquid, the cleaning liquid absorbs ammonia or a chemical compound (connection) of ammonia with further components from the raw gas, thus reducing the ammonia content. For example, the cleaning liquid contains sulfuric acid, and the sulfuric acid and ammonia in the raw gas cause a chemical reaction.

In this implementation of an exhaust gas purification plant, the first monitoring unit comprises a conductivity sensor and/or a pH sensor. The conductivity sensor is able to measure the electrical conductivity of the cleaning liquid and to generate a signal. The signal contains information about the measured electrical conductivity. The measuring position of the conductivity sensor is preferably located downstream of the area where the raw gas is passed through the cleaning liquid. The pH sensor is able to measure the pH value of the cleaning liquid and to generate a signal comprising the measured pH value. Preferably, the measuring position of the pH sensor is located downstream of the exhaust gas purification plant, for example in a container in which the cleaning liquid is collected. The generated signal contains information about the measured pH value.

The two signals are transmitted from the first monitoring unit to the central computer, preferably as part of a message, and are processed by the evaluation unit. According to the present disclosure, the evaluation unit detects any period in which the quality function for the exhaust gas purification plant continuously takes a functional value that is smaller than a specified lower limit. According to the embodiment just described, the evaluation unit additionally detects any period in which at least one of the following two events occurs continuously:
The electrical conductivity of the cleaning liquid is greater than a specified upper limit.
The pH value of the cleaning liquid is smaller than a specified lower limit, i.e., the cleaning liquid is relatively acidic.

The evaluation unit classifies and uses any period with a small quality function and/or a large electrical conductivity and/or a small pH value as a malfunction period.

In some examples, if the electrical conductivity is high, a chemical decomposition process can occur in the cleaning liquid after the cleaning liquid has absorbed the ammonia. As a result, relevant amounts of nitrogen oxides (NOₓ) can escape from the cleaning liquid and enter the environment. A low pH value can also be an indication that nitrogen oxides are entering the environment. Nitrogen oxides entering the environment is an undesirable event. A period in which this undesirable event occurs continuously is therefore also treated as a malfunction period. This embodiment can be combined with a nitrogen oxide sensor but eliminates the need to provide a nitrogen oxide sensor.

An implementation of the just described combination of the two embodiments is described below, namely, that in some examples, an identifier of the applied operating principle is used and, in some examples, the electrical conductivity and the pH value are measured and evaluated. An identifier that distinguishes at least the following two operating principles is stored and evaluated:
The exhaust gas purification plant works purely chemically.
The exhaust gas plant works completely or at least partially biologically.

The problem described above, namely that nitrogen oxides escape at high electrical conductivity or a low pH value, occurs only in a biological exhaust gas purification plant. The evaluation program therefore uses the measured electrical conductivity and the measured pH value only if additionally the identifier that the exhaust gas purification plant works biologically is transmitted.

An embodiment which is described in the following makes it possible to check at least some data connections of the monitoring assembly. According to this embodiment, the monitoring assembly comprises for the or every raw gas sensor and for the or every clean gas sensor of the first monitoring unit a respective signal generator. Every signal generator is configured to capture a value for an ammonia content wherein this value is given to the signal generator by a user and/or by the evaluation unit or a further part of the monitoring assembly. Every signal generator is configured to generate a respective signal wherein the generated signal comprises information about that value for an ammonia content which is given to this signal generator, and which is captured by the signal generator. In one implementation, the generated signal further comprises an identifier of the signal generator wherein this identifier distinguishes this signal generator from the or every other signal generator as well as from every ammonia sensor of the first monitoring unit.

According to this embodiment, the monitoring assembly can selectively be operated in a monitoring mode or in a checking mode. The monitoring method according to a corresponding embodiment comprises the step that at least once a checking method is performed. When being operated in the checking mode and by performing the checking method, the monitoring assembly is checked. When operating in the checking mode, in lieu of the or every raw gas sensor and the or every clean gas sensor of the first monitoring unit the respective signal generator is used. In one implementation, every raw gas sensor and every clean gas sensor is replaced with a respective signal generator. The checking method comprises in one implementation the step that the or every raw gas sensor and the or every clean gas sensor is replaced with a respective signal generator, or the checking method is performed after this replacement is done. It is also possible that selectively an ammonia sensor or a signal generator is activated, namely depending on the mode in which the monitoring assembly is currently operated.

When being operated in the checking mode, the monitoring assembly is configured to perform the following steps, and the checking method comprises the following steps:
Every signal generator generates a respective signal. This signal comprises information on the value of the ammonia content which is given for this signal generator, and which is captured by the signal generator.
The respective signal of every signal generator is transmitted to the evaluation unit.
The evaluation unit determines for every signal generator of the first monitoring unit which value of an ammonia content is given to this signal generator. For doing so, the evaluation unit uses the received signals of the signal generators.
Preferably, the evaluation unit compares for every signal generator the actually given value with the value determined by using the transmitted and received generator signals.

In particular, this embodiment facilitates to detect the following possible faults and malfunctions:
A data connection between an ammonia sensor and thereby a signal generator on the one hand and the evaluation unit on the other hand is interrupted or disturbed in an alternative way.
A signal processing unit installed on the path from an ammonia sensor and thereby from a signal generator to the evaluation unit operates faulty or has a failed.
A part of the monitoring assembly is not at all or not sufficiently supplied with electrical energy.

In one implementation, a respective temporal course of values of an ammonia content is given to at least one signal generator, preferably to every signal generator. With other words: Subsequently different values for an ammonia content are given to at least one signal generator. In one implementation, a test pattern with values for the ammonia content is given to at least one, preferably to every signal generator. Preferably, the temporal course comprises on the one hand the value 0% and on the other hand the maximum possible value for an ammonia content which the respective replaced ammonia sensor can measure and or which can occur in reality. Preferably, the maximum value is between 90% and 100%.

The embodiment with the given temporal courses facilitates to automatically distinguish the monitoring mode from the checking mode, in particular if the given temporal course can in reality not occur while monitoring the exhaust gas purification plant. On the other hand, this embodiment facilitates or at least makes it easier to detect the following malfunctions:
The signal processing unit operates faulty for some values for an ammonia content.
A signal which is generated by a signal generator and thereby generated by the respective ammonia sensor is transmitted to the evaluation unit with a relevant temporal delay.

The present disclosure further relates to a system, wherein this system comprises at least one exhaust gas purification plant, optionally multiple exhaust gas purification plants, and a monitoring assembly according to the present disclosure. The monitoring assembly is capable of monitoring the or each exhaust gas purification plant of the system. To each monitored exhaust gas purification plant a respective monitoring unit of the monitoring assembly is assigned.

### BRIEF DESCRIPTION OF THE DRAWINGS

In the following, the present disclosure is described on the basis of an exemplary embodiment. In the drawings,

FIG. 1 schematically shows an example embodiment of the monitoring assembly according to the present disclosure with a first monitoring unit, which comprises two raw gas sensors and two clean gas sensors;

FIG. 2 schematically shows an example embodiment with the first and a further monitoring unit, which comprises one raw gas sensor and two clean gas sensors;

FIG. 3 is a cross-sectional view of a portion of an example embodiment of the ammonia sensor;

FIG. 4 is a perspective view of the ammonia sensor of FIG. 3;

FIG. 5 shows the principle of the electrochemical sensor cell;

FIG. 6 is a side view of an example assembly with a raw gas sensor, a clean gas sensor, and further sensors;

FIG. 7 shows an example of the respective temporal course of the signal of a raw gas sensor and of two clean gas sensors;

FIG. 8 shows an example display of a monitoring result over a period of time;

FIG. 9 shows an example of respective monitoring results represented in six periods of time;

FIG. 10 shows an example of a summary representation availability rate of an exhaust gas purification plant over a period of twelve months; and

FIG. 11 shows the embodiment of FIG. 1 with signal generators in lieu of ammonia sensors.

### DETAILED DESCRIPTION

In an example embodiment, the present disclosure is applied to an exhaust gas purification plant in a building. Such an exhaust gas purification plant is capable of removing ammonia (NH₃) from a gas mixture. In the exemplary embodiment, the gas mixture comes from an animal breeding facility. The gas mixture can also be created, for example, in a plant for the treatment of sewage sludge or in a production plant for certain chemical compounds or for foodstuffs. Before purification, the gas mixture typically contains an ammonia content of between 1 and 1000 ppm (parts per million).

According to some examples, such an exhaust gas purification plant works biologically. Microbes decompose the ammonia in the gas mixture. According to another embodiment, the exhaust gas purification plant works according to a chemical operating principle. This generally means that the gas mixture is subjected to a chemical reaction, in particular by adding a chemical substance to the gas mixture. The chemical reaction converts the ammonia, and the result of the conversion provides substances that are less harmful to humans than ammonia, or even harmless.

One implementation of an active chemical principle is the following: The gas mixture containing ammonia is brought into contact with sulfuric acid (H₂SO₄) in the chemical exhaust gas purification plant. The chemical reaction H₂SO₄+2 NH₃→(NH₄)₂SO₄ forms ammonium sulfate. Said ammonium sulfate is not gaseous at normal ambient temperatures and can be used as a fertilizer or as a nutrient for microbes.

Hereinafter, the terms “raw gas” and “clean gas” will be used. The raw gas is fed to the exhaust gas purification plant, and clean gas leaves the exhaust gas purification plant. The raw gas contains ammonia or can at least contain ammonia. If the exhaust gas purification plant is working properly, the ammonia content in the clean gas is significantly lower than in the raw gas. Ideally, the clean gas contains no ammonia at all.

More precisely, “significantly lower” means that the exhaust gas purification plant is working properly if a specified quality function for the exhaust gas purification plant is above a specified lower limit. A quality function and a lower limit for the quality function are thus specified in a computer-evaluable form. In some examples the quality function increases as the ammonia content in the clean gas decreases relative to a constant ammonia content in the raw gas. For example, the quality function may be equal to a purification efficiency Rg=Rg(Ammᵣₑᵢₙ, Ammᵣₒₕ)=1−Ammᵣₑᵢₙ/Ammᵣₒₕ, where Ammᵣₑᵢₙ is the ammonia content in the clean gas and Ammᵣₒₕ is the ammonia content in the raw gas. In an alternative example, a purification efficiency Rg=Δ/Ammᵣₒₕ is used as a quality function, where Δ=Ammᵣₒₕ−Ammᵣₑᵢₙ. In both examples, the quality function takes a value of between 0 and 1, wherein the value 1 means that the exhaust gas purification plant has completely removed the ammonia from the raw gas, i.e. Ammᵣₑᵢₙ=0.

The present disclosure automatically monitors the exhaust gas purification plant to determine whether or not it is working properly in the sense of the above definition. The assembly according to the present disclosure comprises m raw gas sensors and n clean gas sensors. Here, m≥1 and n≥1 are two numbers that may be the same or different. The sensors can all work according to the same measuring principle or apply at least two different measuring principles. The or each raw gas sensor measures the ammonia content in the raw gas and is therefore arranged upstream of the exhaust gas purification plant. The or each clean gas sensor measures the ammonia content in the clean gas and is therefore arranged downstream of the exhaust gas purification plant. In many cases, it is advantageous to use at least two raw gas sensors and/or at least two clean gas sensors, in particular for the following reasons:
The ammonia content in the raw gas or the ammonia content in the clean gas at a given time can vary in space.
A sensor can fail completely.
A sensor can provide a measured value with a significant measurement malfunction, usually a measured value that is too small.

The present disclosure can also be implemented with only one raw gas sensor and/or only one clean gas sensor.

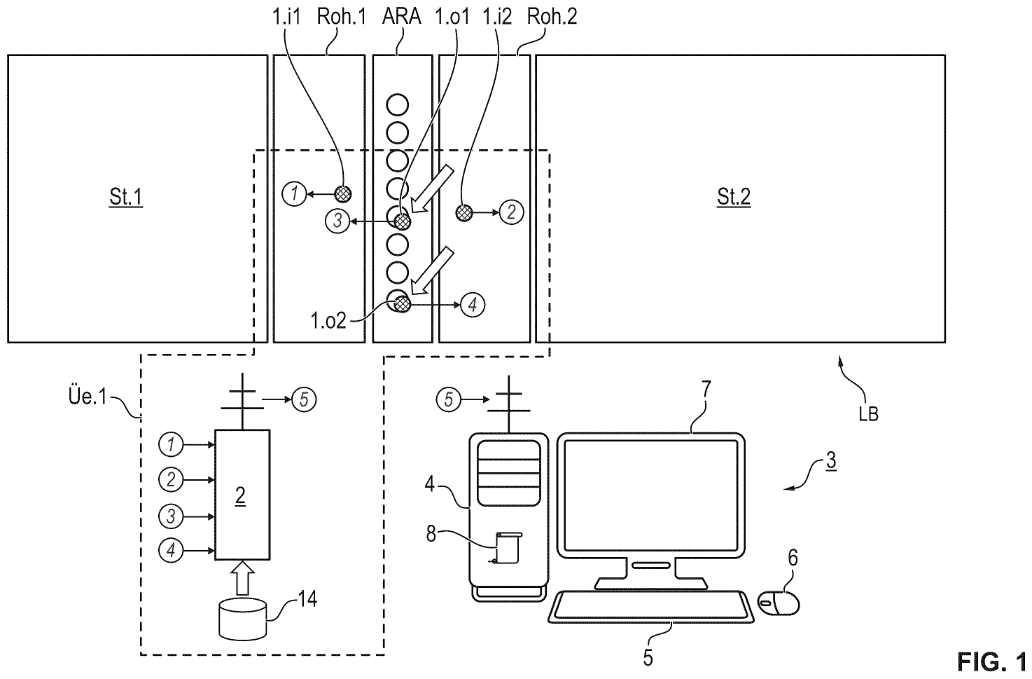

FIG. 1 schematically shows an example of an embodiment of the monitoring assembly according to the present disclosure with a first monitoring unit Ue.1 and a central computer 3. Components of the monitoring assembly according to the present disclosure are designated with numbers, and other objects are designated with letters.

In some examples, the exhaust gas purification plant ARA is used in an agricultural enterprise LB, which comprises two stables St.1, St.2. Animals are kept in both stables St.1 and St.2, and ammonia can therefore be produced in each stable St.1, St.2. The first monitoring unit Ue.1 is assigned to and monitors the exhaust gas purification plant ARA.

In a region called Roh.1, raw gas escaping from the stable St.1 can accumulate. Accordingly, raw gas escaping from the stable St.2 can accumulate in a region Roh.2. The exhaust gas purification plant ARA is capable of removing ammonia from both the raw gas of the Roh.1 region and the raw gas of the Roh.2 region.

A first raw gas sensor 1.i1 is arranged in the region Roh.1 and is able to measure the ammonia content in the gas mixture present in the region Roh.1. Accordingly, a second raw gas sensor 1.i2 is arranged in the region Roh.2 and is able to measure the ammonia content in the gas mixture present in the region Roh.2. The two raw gas sensors 1.i1, 1.i2 measure the ammonia content at two different measuring positions, and the ammonia content can vary not only over time but also from measuring position to measuring position.

Two clean gas sensors 1.o1, 1.o2 are arranged downstream of the exhaust gas purification plant ARA. These sensors measure the ammonia content in the clean gas at two spaced-apart measuring positions.

Each sensor 1.i1, 1.i2, 1.o1, 1.o2 generates a respective signal, which contains information about the ammonia content measured by the sensor 1.i1, 1.i2, 1.o1, 1.o2. The ammonia content is preferably stated in ppm (parts per million), alternatively in vol % (percent by volume) or wt % (percent by weight). The sensors 1.i1, 1.i2, 1.o1, 1.o2 transmit their signals to a communication unit 2, which acts as the first communication unit. In some examples, the communication unit 2 regularly queries the sensors 1.i1, 1.i2, 1.o1, 1.o2, and in response to a query, the queried sensor transmits at least one signal value, unless the queried sensor is defective, or the data connection is interrupted. The communication unit 2 is also arranged in the agricultural enterprise LB. The signals are transmitted via cable and/or via radio waves from the sensors 1.i1, 1.i2, 1.o1, 1.o2 to the communication unit 2. The sensors and the communication unit 2 belong to the first monitoring unit Ue.1, which is assigned to the agricultural enterprise LB having the exhaust gas purification plant ARA.

The communication unit 2 has, at least temporarily, read access to a data memory 14. Information about the first monitoring unit Ue.1 and information about each sensor of the first monitoring unit Ue.1 are stored in said data memory 14.

In some examples, the following information is stored in the data memory 14:
a unique identifier (ID) of the communication unit 2 and thus a unique identifier of the assigned exhaust gas purification plant ARA to be monitored, wherein the identifier ID distinguishes said communication unit 2 from any other communication unit, and
a unique identifier b or c or bc that specifies which cleaning (operating) principle is applied by the monitored exhaust gas cleaning plant ARA, for example whether it removes ammonia biologically or chemically or in both ways.

In some examples, a respective unique identifier is stored for each sensor 1.i1, 1.i2, 1.o1, 1.o2. This unique identifier comprises the following components:
a unique identifier i1, i2, o1, o2 for the sensor, wherein the unique identifier distinguishes this sensor 1.i1, 1.i2, 1.o1, 1.o2 from any other sensor in the same enterprise LB, and
in some examples, whether the sensor measures clean gas or raw gas, i.e., whether it is arranged upstream or downstream of the monitored exhaust gas purification plant ARA.

In the exemplary embodiment of FIG. 1, the monitoring assembly according to the present disclosure also comprises a central computer 3, which comprises the following components:
a signal-processing computing unit 4,
a screen 7,
a keyboard 5, and
a mouse 6.

The central computer 3 is arranged at a distance from the enterprise LB, in which the assigned and monitored exhaust gas purification plant ARA is located. The communication unit 2 transmits the signals from the sensors 1.i1, 1.i2, 1.o1, 1.o2 via cable and/or via radio waves to the central computer 3.

Each transmitted signal contains the information described above, i.e.,
the unique identifier of the communication unit 2,
the number m, n of sensors of the exhaust gas purification plant ARA,
the operation principle of the exhaust gas purification plant ARA, and
the above-described unique identifier of the sensor 1.i1, 1.i2, 1.o1, 1.o2 from which the measured ammonia content in the signal originates.

The communication unit 2 determines this information by having read access to the data memory 14.

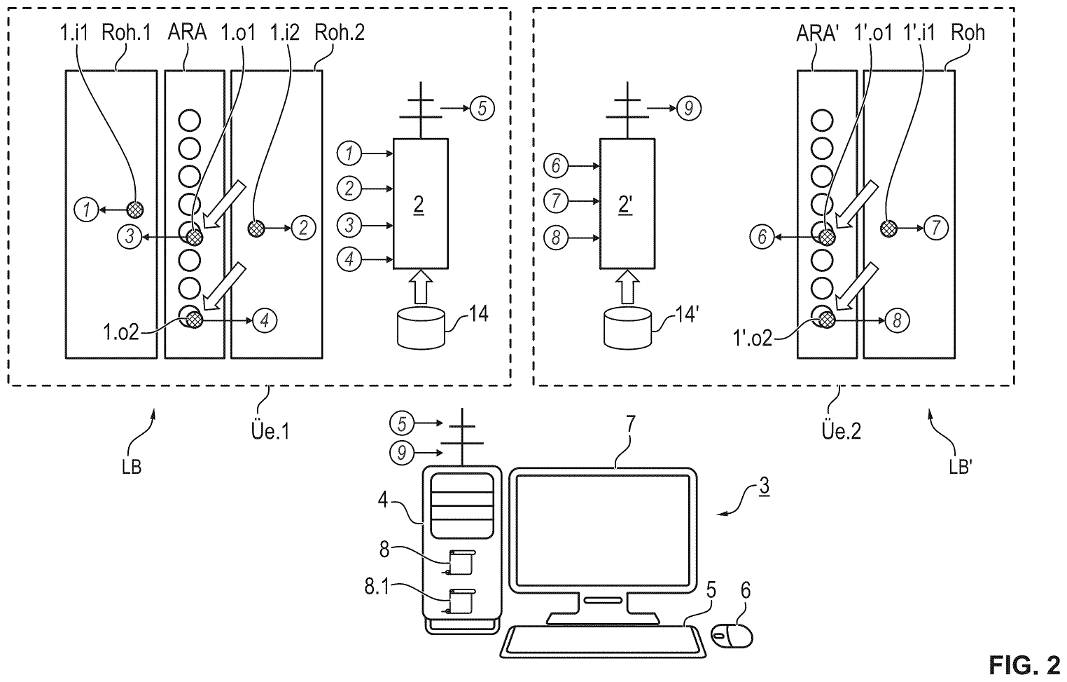

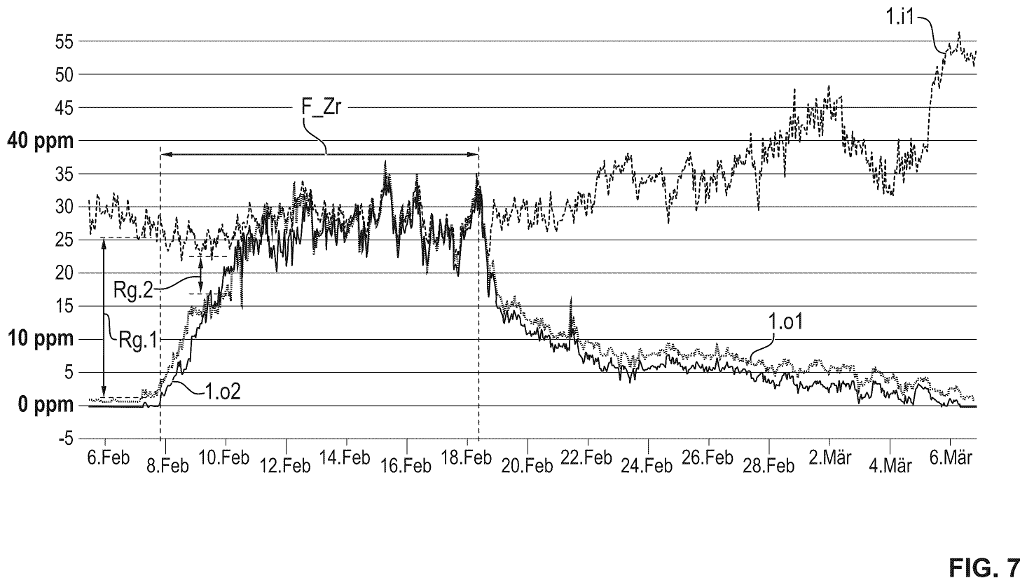

The computing unit 4 comprises a processor and a data memory. An evaluation program 8 is stored on the data memory. The processor is capable of executing the evaluation program 8 and of processing signals during execution. The central computer 3 received these signals from the first monitoring unit Ue.1 and optionally from further monitoring units, (e.g., as illustrated in FIG. 2). The processor is able to generate different representations depending on the signals processed. Signal processing and the representations are described below with reference to FIGS. 7 to 10.

In the exemplary embodiment, the evaluation unit is installed on the computing unit 4 and comprises the evaluation program 8. It is also possible for the evaluation unit or at least part of the evaluation unit to be a component of the communication unit 2.

FIG. 2 shows the exhaust gas purification plant ARA, the first monitoring unit Ue.1 of FIG. 1 as well as a further exhaust gas purification plant ARA′ and a further monitoring unit Ue.2 in a further agricultural enterprise LB′. The further exhaust gas purification plant ARA′ removes ammonia from raw gas, which can accumulate in a further region Roh. The further monitoring unit Ue′.2 comprises a further raw gas sensor 1′.i1, two further clean gas sensors 1′.o1, 1′.o2, and a further communication unit 2′. A respective data connection between a further sensor 1′.i1, 1′.o1, 1′.o2 and the further communication unit 2′ is established at least temporarily. In addition, a data connection is established at least temporarily between the further communication unit 2′ and the central computer 3, wherein the central computer 3 has already been described with reference to FIG. 1.

In the example shown in FIG. 2, the central computer 3 receives signals from the first communication unit 2 as well as signals from the further communication unit 2′. Each communication unit 2, 2′ belongs to a respective monitoring unit Ue.1, Ue.2, wherein said monitoring unit Ue.1, Ue.2 further comprises m respective raw gas sensors and n respective clean gas sensors. The following applies: 1≤m≤M and 1≤n≤N, where M and N are specified maximum numbers of sensors and, for example, M=N=4. The numbers m and n can vary from monitoring unit Ue.1 to monitoring unit Ue.2. The evaluation program 8 on the computing unit 4 comprises M*N variants, namely, one respective variant for a monitoring unit with m raw gas sensors and n clean gas sensors, where 1≤m≤M and 1≤n≤N.

If the evaluation depends on which operating principle is used by the monitored exhaust gas purification plant, and if there are K different operating principles, M*N*K different variants of the evaluation program are provided.

As an example, FIG. 2 shows an evaluation program 8.1 in addition to evaluation program 8. The evaluation program 8 is intended for one type of exhaust gas purification plants, for example for biological plants, and the evaluation program 8.1 is intended for a second type, for example for chemical plants. Each evaluation program 8, 8.1 comprises M*N variants.

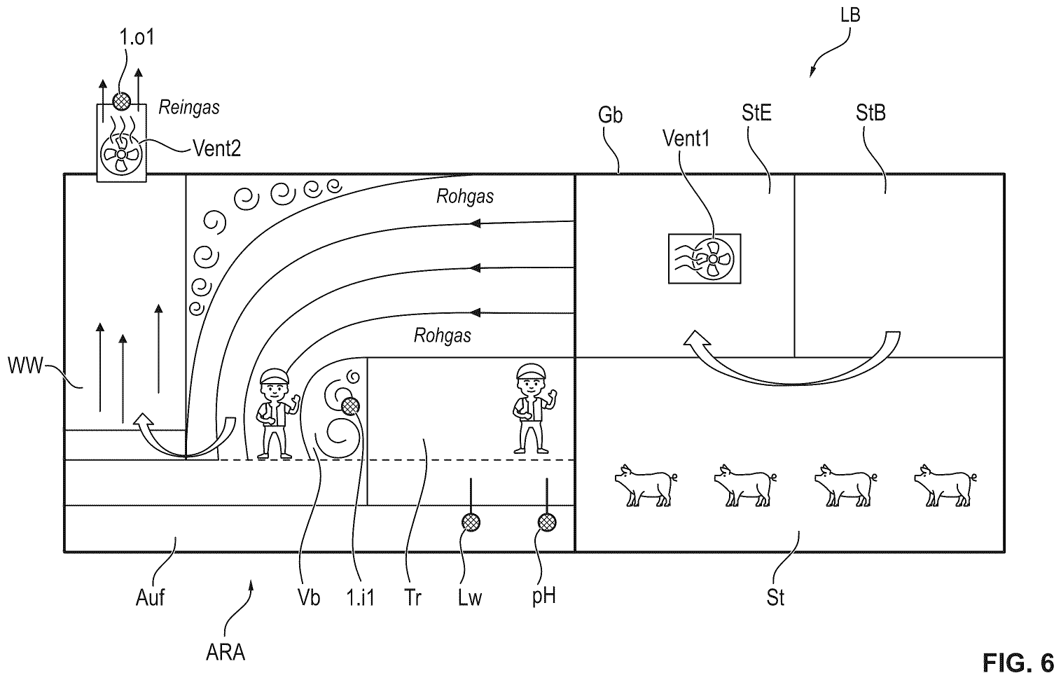

In another implementation, the evaluation program 8 comprises two variants each for a monitoring unit with m raw gas sensors and n clean gas sensors, namely, one variant for an exhaust gas purification plant that removes ammonia solely chemically and one variant for an exhaust gas purification plant that removes ammonia biologically or both biologically and chemically. The differences between these two variants are explained below with reference to FIG. 6.

As already explained, the processor of the computing unit 4 executes the evaluation program 8. The evaluation program 8 evaluates the signals from each connected communication unit 2, 2′. Through this evaluation, the evaluation program 8 determines the following for each connected monitoring unit Ue.1, Ue.2:
to which exhaust gas purification plant ARA, ARA′ the signals of this monitoring unit Ue.1, Ue.2 relate,
how many raw gas sensors and how many clean gas sensors this monitoring unit Ue.1, Ue.2 has, and
whether the assigned monitored exhaust gas purification plant ARA, ARA′ works biologically, chemically, or both biologically and chemically.

In addition, the evaluation program 8 determines from which sensor a signal containing information about an ammonia content originates.

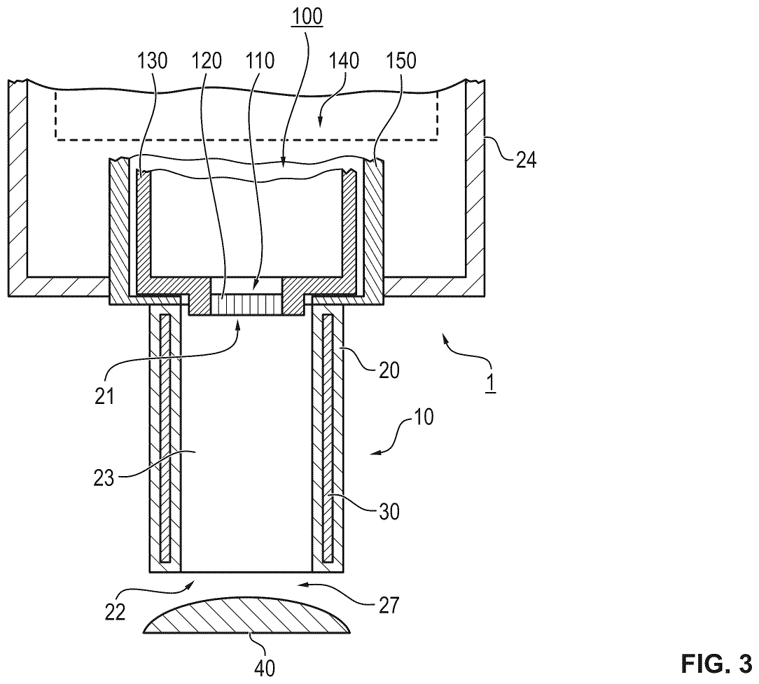

An example embodiment of an ammonia sensor 1 is described below with reference to FIGS. 3 to 5. Both the two raw gas sensors 1.i1, 1.i2 and the two clean gas sensors 1.o1, 1.o2 of FIG. 1 can be constructed as described below. The terms “top” and “bottom” described below relate to an orientation of the ammonia sensor 1 during regular operation.

The ammonia sensor 1 comprises a sensor cell 100 with a measuring chamber. A wall 130 and a porous protective filter 120 surround the sensor cell 100. The sensor cell 100 measures the ammonia content in a gas sample, which is located in the measuring chamber with the wall 130. The sensor cell 100 uses at least one of multiple known measuring principles to measure the ammonia content in a gas mixture.

The gas sample flows from below through an inlet opening 22 into a tubular feed unit 10 and “settles” in a region 23 of the feed unit 10. The settled gas sample flows upward through the feed unit 10 and through an outlet opening 21 into a region 110 of the measuring chamber. This region is delimited by the wall 130, the outlet opening 21, and a membrane 121. Above the membrane 121, an electrolyte and multiple electrodes are located in the measuring chamber. The protective filter 120 is arranged between the feed unit 10 and the region 110 of the measuring chamber.

The feed unit 10 is delimited by a wall 20. A heating element 30 is inserted into the wall 20. The heating element 30 causes a convection flow through the inlet opening 22 upwards into the feed unit 10 (chimney effect). A mechanical impact protection element 40 reduces the risk of the convection flow entraining particles into the feed unit 10. An annular inlet gap 27 is formed between the impact protection 40 and the inlet opening 22. Incoming gas is often swirled in said inlet gap 27, which leads to a smaller spatial variation of the ammonia content in the gas sample. The resulting swirl is therefore desirable.

The ammonia sensor 1 further comprises a housing 24, which surrounds an interior region 140. A power supply unit and a signal-processing evaluation unit are arranged in the interior region 140. Thanks to its own power supply unit, the ammonia sensor 1 is independent of a stationary power supply network. A holder 150 is inserted in the bottom of the housing 24. Said holder 150 holds the feed unit 10 and surrounds the wall 130 of the sensor cell 100.

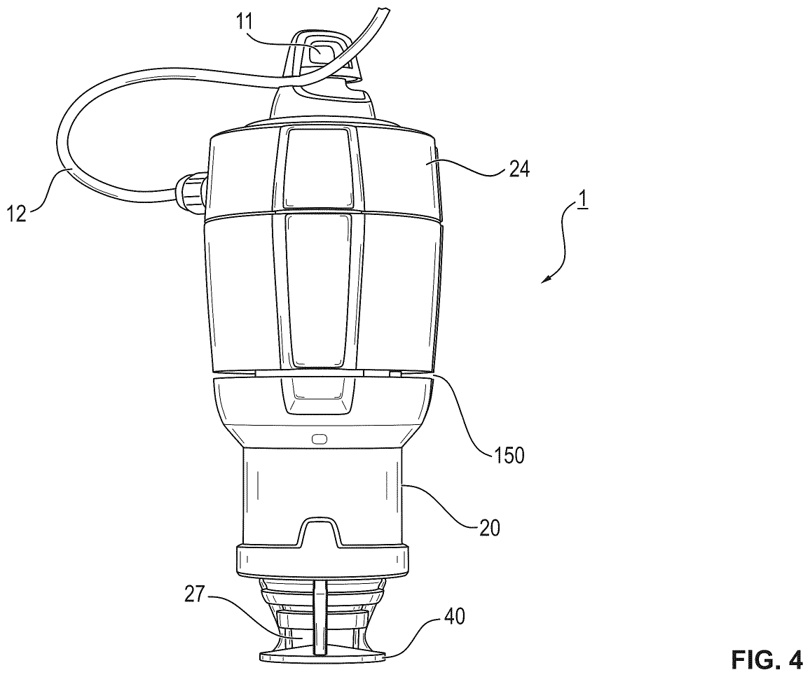

FIG. 4 shows the ammonia sensor 1 of FIG. 3 in a side view. Same reference signs have the same meanings as in FIG. 3. During operation, the ammonia sensor 1 is arranged such that the impact protection 40 points downward. An eyelet 11 is attached to the top of the housing 24 and can be used to hang the ammonia sensor 1 on a hook. In addition, a cable 12 is attached to the housing 24. Thanks to the cable 12, the ammonia sensor 1 can be connected to a stationary power supply network, for example to charge its own power supply unit.

The or each raw gas sensor 1.i1, 1.i2 is preferably arranged at a position in which the raw gas is swirled. For example, the raw gas flows almost horizontally over a surface, reaches an edge and then flows vertically or obliquely downward. This results in a swirl. Because the raw gas is swirled at the measuring position of the raw gas sensor 1.i1, 1.i2, the raw gas sensor 1.i1, 1.i2 measures an ammonia content that is spatially averaged to a certain extent.

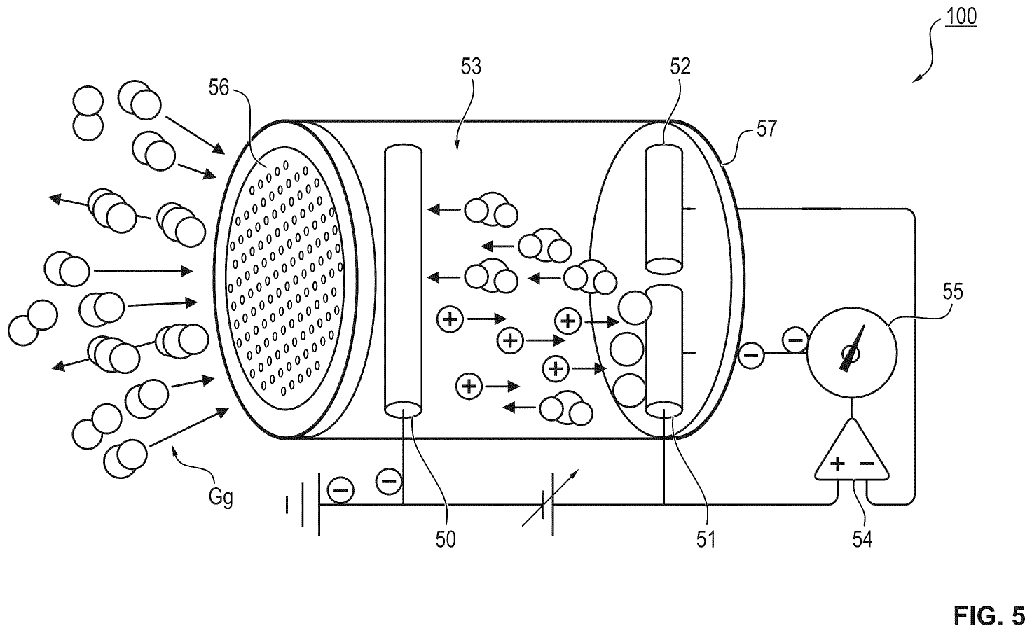

FIG. 5 schematically illustrates the mode of operation of an electrochemical sensor cell 100 of an ammonia sensor 1. The sensor cell 100 works according to the principle of a fuel cell. The ammonia content in a gas mixture Gg is to be investigated. The gas mixture Gg flows through a porous membrane 56 into a measuring chamber inside a housing 57. A measuring electrode 50, a counter electrode 51, and a reference electrode 52 are located inside the housing. An ionically conductive electrolyte 53 is located between the measuring electrode 50 on one side and the counter electrode 51 and the reference electrode 52 on the other side. The gas mixture Gg reaches the electrolyte 53. As shown schematically, an electric current flows in the electrolyte 53. A potentiostat 54 measures an indicator for the total flowing electrical charge. The electrical charge correlates with the amount of ammonia in the measuring chamber and thus with the ammonia content in the gas mixture Gg, wherein the gas mixture Gg is located to the left of the membrane 56. The measured ammonia content can be read on a display 55.

The or each clean gas sensor 1.o1, 1.o2 is preferably located obliquely above the exhaust gas purification plant ARA, for example on a roof of a building in which the exhaust gas purification plant ARA is located. In some examples, a fan or other fluid conveying unit extracts the clean gas from said building. The or a clean gas sensor 1.o1, 1.o2 is located above this fluid conveying unit and preferably outside and above the building. This positioning makes it easier to service and repair the clean gas sensor 1.o1, 1.o2.

Preferably, a fluid-impermeable tube surrounds each sensor 1.i1, 1.i2, 1.o1, 1.o2 or at least each sensor arranged outside the building. The longitudinal axis of this tube is arranged vertically, i.e., approximately in parallel with the convection flow generated by the heating element 30. This tube protects the sensor 1.i1, 1.i2, 1.o1, 1.o2 to a certain extent from external mechanical influences. In addition, the tube protects the sensor 1.i1, 1.i2, 1.o1, 1.o2 from the influence of wind and other air flows. Thanks to the tube, sufficient convection flow is generated even in the case of air flows. The risk of air flows causing measurement malfunctions is reduced.

FIG. 6 shows a side view of an example of an agricultural enterprise LB with a stable St and a chemical exhaust gas purification plant ARA, both of which are arranged in a building Gb. Four pigs are shown schematically in the stable St. The stable St is aerated in a region StB and de-aerated in a region StE by means of a first fan Vent1. Raw gas from the stable St, which usually contains a relatively high ammonia content, flows from the region StE almost horizontally over the roof of a technical room Tr and then flows down a wall of the technical room Tr. A swirl region Vb is formed at said wall. Thanks to said swirl region Vb, the ammonia content varies less in space at a point in time downstream of the swirl region Vb.

A second fan Vent2 on the roof of building Gb extracts clean gas from the building Gb upward. A cleaning liquid is applied from above onto a scrubber wall WW and flows down the scrubber wall WW. At the scrubber wall WW, sulfuric acid (NH₃) is added to the raw gas passing by, for example as part of the cleaning liquid. The addition of sulfuric acid causes the chemical reaction H₂SO₄+2 NH₃→(NH₄)₂SO₄ described above. A collection container Auf obliquely below the scrubber wall WW collects the cleaning liquid containing the ammonium sulfate (NH₄)₂SO₄.

A pH sensor pH measures the pH value of the cleaning liquid in the collection container Auf. A conductivity sensor LW measures the electrical conductivity of the cleaning liquid in the collection container Auf. One background is the following limitation of a biological exhaust gas purification plant ARA: If the electrical conductivity of the cleaning liquid in the collection container Auf is greater than an upper limit or the pH value is lower than a lower limit, the exhaust gas purification plant ARA may not work properly. Chemical decomposition or degradation processes may occur in the cleaning liquid in the collection container Auf, and nitrogen oxide (NOₓ) can escape as a result thereof. This is undesirable. The upper limit for the electrical conductivity is preferably less than 300 mS/cm and is particularly preferably between 5 and 50 mS/cm (mS=milli Siemens), in particular between 10 and 35 mS/cm. The lower limit for the pH value is preferably between 5 and 8.

Preferably, the cleaning liquid containing the ammonium sulfate is regularly extracted from the collection container Auf. In one implementation, the ammonium sulfate is at least partially removed from the cleaning liquid by a chemical process, for example by targeted decomposition, and the cleaning liquid is returned to the scrubber wall WW. In another implementation, the cleaning liquid is used for another purpose or disposed of, and new cleaning liquid is fed to the exhaust gas purification plant.

Preferably, a closed-loop control is used to ensure that the pH value of the cleaning liquid fed to the scrubber wall WW is within a specified range. The pH sensor pH measures the actual pH value of the cleaning liquid that is fed to the exhaust gas purification plant. If necessary, the pH value of the supplied cleaning liquid is increased or decreased.

In this example, the monitoring unit comprises a raw gas sensor 1.i1 arranged in the swirl region Vb and a clean gas sensor 1.o1 arranged above the second fan Vent2 and thus outside the building Gb and on the roof. The clean gas sensor 1.o1 is surrounded by a vertically arranged tube (not shown). In some examples, the communication unit 2 receives a signal of the pH sensor pH and a signal of the conductivity sensor LW and forwards these two signals.

The central computer 3 captures a user input made by a user using the keyboard 5 and/or the mouse 6. Said user input specifies which evaluations the central computer 3 should generate and display. The central computer 3 captures the user input and generates at least one representation according to the captured user input. The central computer 3 causes the desired representation to be output on the screen 7 in a form that can be perceived by a human. In order to generate said representation, the central computer 3 receives signals from the connected monitoring units Ue.1, Ue.2.

The evaluation program 8 evaluates the signals from the respective communication unit 2, 2′ of the monitoring units Ue.1, Ue.2. As explained above, the evaluation program 8 comprises a total of 2*M*N variants, wherein M is the maximum possible number of raw gas sensors and N the maximum possible number of clean gas sensors and there is also one respective variant for a purely chemical exhaust gas purification plant and for an exhaust gas purification plant that works solely or at least partially biologically.

As mentioned above, the identifier of a monitoring unit comprises an identifier indicating how the monitored exhaust gas purification plant ARA, ARA′ works, namely, in the implementation shown, “b” or “c” or “bc”.

The variant for a chemical exhaust gas purification plant preferably also evaluates a transmitted signal of a sensor for the electrical conductivity and a transmitted signal of a sensor for the pH value. With this evaluation, the variant of the evaluation program 8 determines the respective electrical conductivity and the respective pH value and compares these two values with a respective limit explained above. Preferably, the variant for a biological exhaust gas purification plant does not carry out these comparisons.

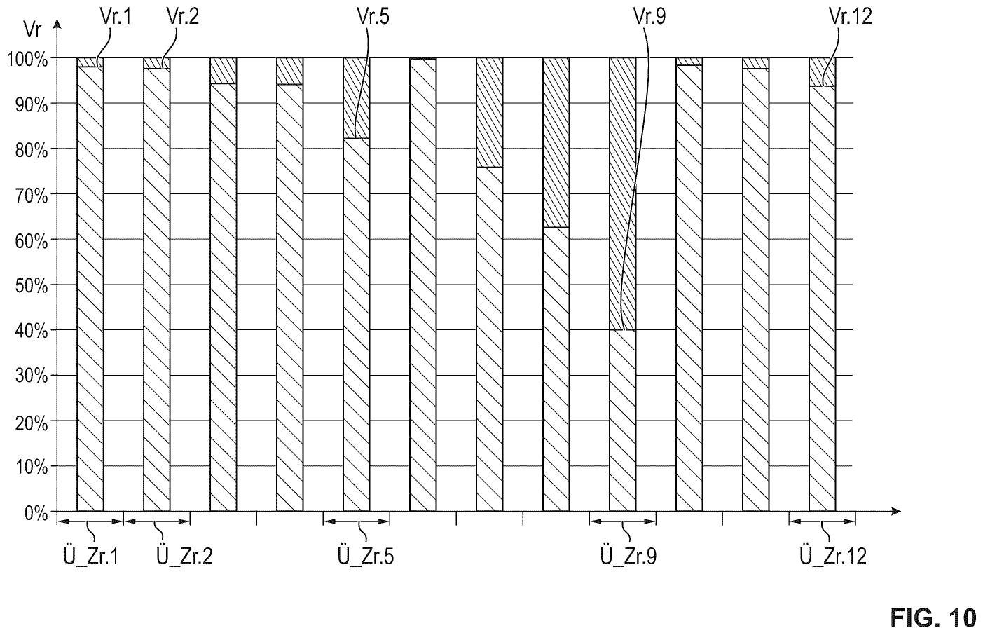

FIGS. 7 to 10 show different representations, which differ from one another with regard to the respective monitoring period U_Zr and the level of detail. In each representation, the x-axis represents time and the y-axis represents the ammonia content (FIGS. 7 to 9) or an availability rate of the monitored exhaust gas purification plant (FIG. 10).

FIG. 7 relates to a monitoring period U_Zr of one month duration. Shown are the respective temporal course of the ammonia content measured by the raw gas sensor 1.i1 and the clean gas sensors 1.o1 and 1.o2, cf. FIG. 2, i.e., a total of three different temporal courses. The evaluation program 8 also calculates a quality function in the form of a purification efficiency Rg=Rg(Ammᵣₑᵢₙ/Ammᵣₒₕ) for each sampling time, for example according to the following calculation rule:

$$Rg = 1 - {Amm}_{rein} / {Amm}_{roh} \qquad ( 1 )$$

$$or$$

$$Rg = ( {Amm}_{roh} - {Amm}_{rein} ) / {Amm}_{roh} . \qquad ( 2 )$$

The evaluation program 8 determines each malfunction period. During a malfunction period, the purification efficiency Rg is below a specified lower limit. The lower limit is at most equal to 1 and is preferably greater than 0.01, particularly preferably greater than 0.6, and is for example 0.9. In the case of a chemical exhaust gas purification plant, a malfunction period is also a period in which the electrical conductivity is above the specified upper limit and/or the pH value is below the specified lower limit, even if the purification efficiency Rg is sufficiently high.

In the example of FIG. 7, the purification efficiency Rg is continuously below 0.9 in the period F_Zr and is greater than or equal to 0.9 outside the period. Therefore, the period F_Zr is the only malfunction period of the monitoring period U_Zr, which here has a duration of one month.

If a monitoring unit Ue.1, Ue.2 has multiple raw gas sensors, the evaluation program 8 determines the ammonia content in the raw gas by averaging, e.g., averaging in a weighted manner, the measured values provided by the raw gas sensors of said monitoring unit Ue.1, Ue.2. The same applies to a monitoring unit Ue.1, Ue.2 with multiple clean gas sensors.

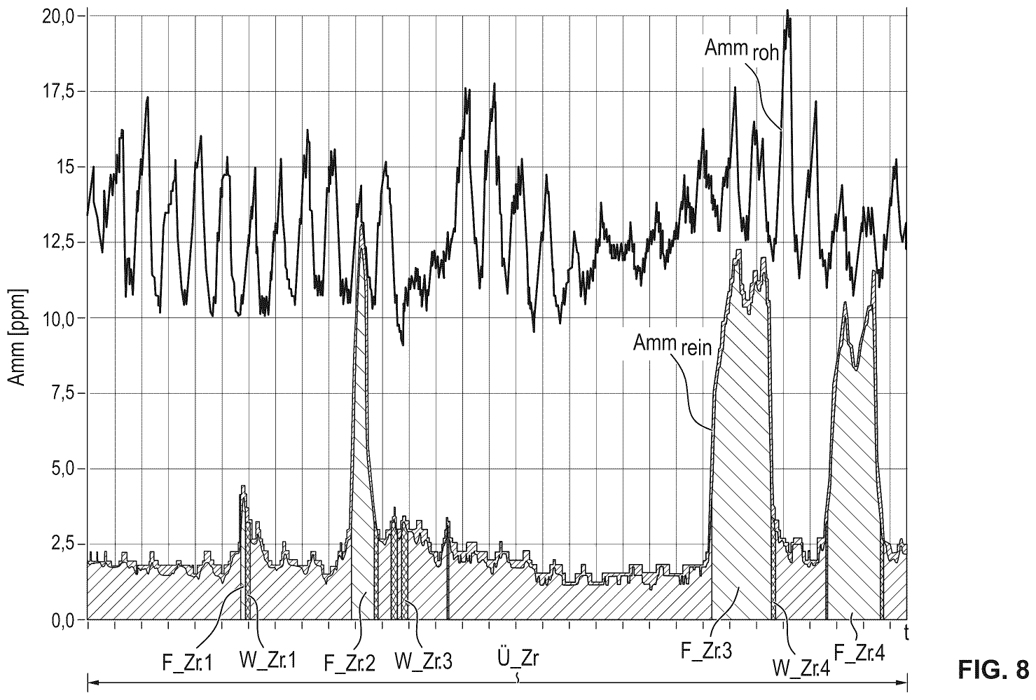

The representation in FIG. 8 shows the temporal course of an averaged ammonia content Ammᵣₒₕ in the raw gas and the temporal course of an averaged ammonia content Ammᵣₑᵢₙ in the clean gas. In this monitoring period U_Zr, four malfunction periods F_Zr.1, . . . , F_Zr.4 were discovered, in which the purification efficiency Rg is continuously smaller than the specified limit of, for example, 0.9. In addition, multiple warning periods W_Zr1, W_Zr2, . . . were detected, in which the purification efficiency Rg is greater than 0.9 but smaller than a further lower limit.

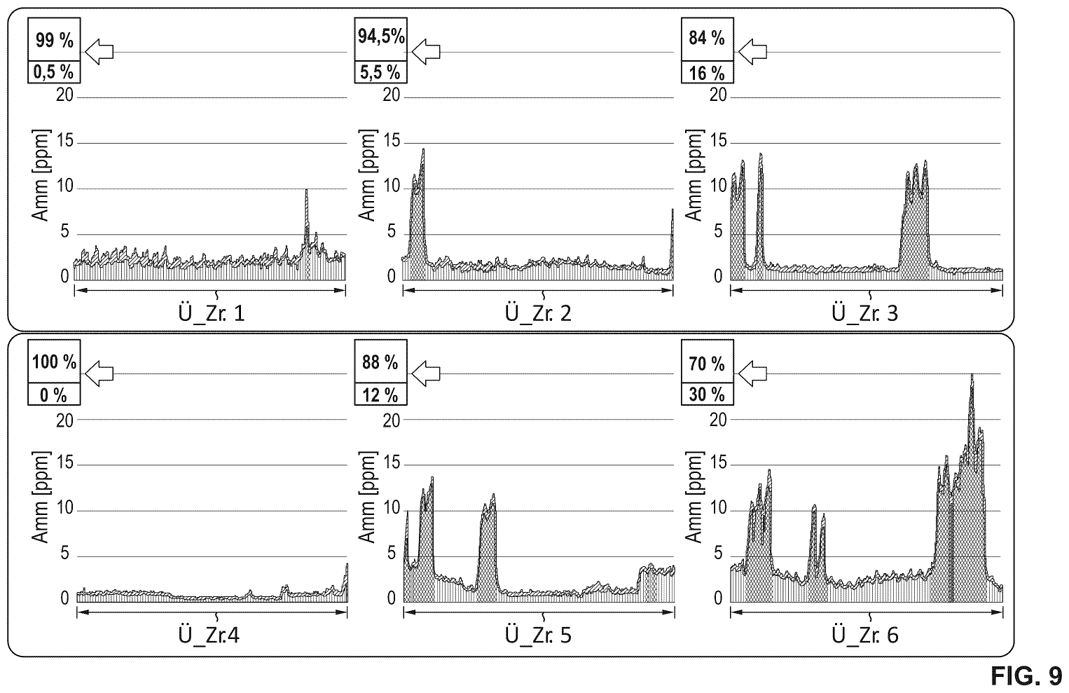

FIG. 9 shows the evaluation for six monitoring periods U_Zr.1, . . . , U_Zr.6 in a single representation. Each individual representation is structured as shown in FIG. 8.

FIG. 10 shows an example of how the respective availability rate Vr.1, . . . , Vr.12 of an exhaust gas purification plant ARA is represented and displayed in a monitoring period. Twelve monitoring periods U_Zr.1, . . . , U_Zr.12 are shown, which each have a duration of one month, for example. The evaluation program 8 calculates the availability rate Vr.i of the exhaust gas purification plant ARA in the monitoring period U_Zr.i according to the following calculation rule:

$$Vr . i = 1 - \{ dur [ F\_Zr \text{.1} ] + … + dur [ F\_Zr . k ( i ) ] \} / d ur ( U\_Zr . i ) , \qquad ( 3 )$$

where F_Zr.1, . . . F_Zr.k(i) are the malfunction periods that occur in the monitoring period U_Zr.i, dur[F_Zr] is the duration of a malfunction period F_Zr, and dur(U_Zr.i) is the duration of the monitoring period U_Zr.i. The number k(i) of malfunction periods may vary from monitoring period to monitoring period and can also be 0.

In some examples, an average availability rate Vr.avg for the last 12 months is derived from the last 12 availability rates Vr.1, . . . , Vr.12. The average availability rate Vr.avg is calculated as a weighted average, i.e., according to the following calculation rule:

$$Vr . avg = α \text{.1} * Vr \text{.1} + … + α \text{.12} * Vr \text{.12} . \qquad ( 4 )$$

In some examples, the weighting factors α.1, . . . , α.12 are determined and specified in particular on the basis of the boundary condition that the volume flow out of a stable in an agricultural enterprise is usually greater in summer than in winter. It is also possible that all weighting factors are equal, i.e., that an arithmetic mean is formed.

It is possible that a raw gas sensor and/or a clean gas sensor may work incorrectly or may have even failed completely. Usually, a faulty sensor will provide an ammonia content that is too low, a failed sensor will even provide an ammonia content of 0, but not an ammonia content that is too high.

The evaluation program 8 on the computing unit 4 or an evaluation program of the monitoring unit Ue.1, Ue.2 can in some cases automatically detect a malfunction and/or a failure of a sensor and can compensate to a certain extent. The following events indicate a sensor fault:
The or a clean gas sensor of a monitoring unit Ue.1, Ue.2 provides a higher ammonia content than the or a raw gas sensor of this monitoring unit Ue.1, Ue.2, and there is a time span between the two sampling times of these two sensors that is smaller than a specified upper limit. In this case, the raw gas sensor is usually defective or faulty. The time span is taken into account because the ammonia content in the raw gas can in fact decrease over a longer time period.
The temporal course of the ammonia concentration in the clean gas and/or in the raw gas decreases faster than a specified change limit. This is often an indication that the sensor that measured this ammonia concentration has become defective. The change limit is chosen such that a decrease that is faster than the change limit cannot correspond to the actual course of an ammonia concentration.
A monitoring unit Ue.1, Ue.2 comprises two raw gas sensors. At one sampling time point, the absolute or percentage deviation between the two measured values, i.e., between the two measured ammonia contents, of these two raw gas sensors is greater than a specified lower limit. Or the absolute or percentage deviation increases more than a specified lower limit. This is an indication that the raw gas sensor that provides the lower ammonia content is working incorrectly or has even failed completely.
The same applies to a monitoring unit Ue.1, Ue.2 with two clean gas sensors.

The evaluation program 8 responds as follows to the detection that a sensor is faulty:
If a monitoring unit Ue.1, Ue.2 has only a single raw gas sensor and said raw gas sensor has failed, the evaluation program 8 generates an error message. The same applies if a monitoring unit Ue.1, Ue.2 has only a single clean gas sensor and said sensor has failed.
If a monitoring unit Ue.1, Ue.2 has two raw gas sensors and one of them measures incorrectly or has failed, the evaluation program 8 uses the measured value of the sensor that measures a higher ammonia content. The same applies to a monitoring unit Ue.1, Ue.2 with two clean gas sensors. For, a malfunction or failure of a sensor usually leads to a measured value of the ammonia content that is too low, but not to a measured value that is too high.
If a monitoring unit Ue.1, Ue.2 comprises two raw gas sensors and these two sensors are intact, the evaluation program 8 determines the ammonia content in the raw gas by averaging, e.g., averaging in a weighted manner, the measured values of these two raw gas sensors. The same applies to a monitoring unit with two clean gas sensors.

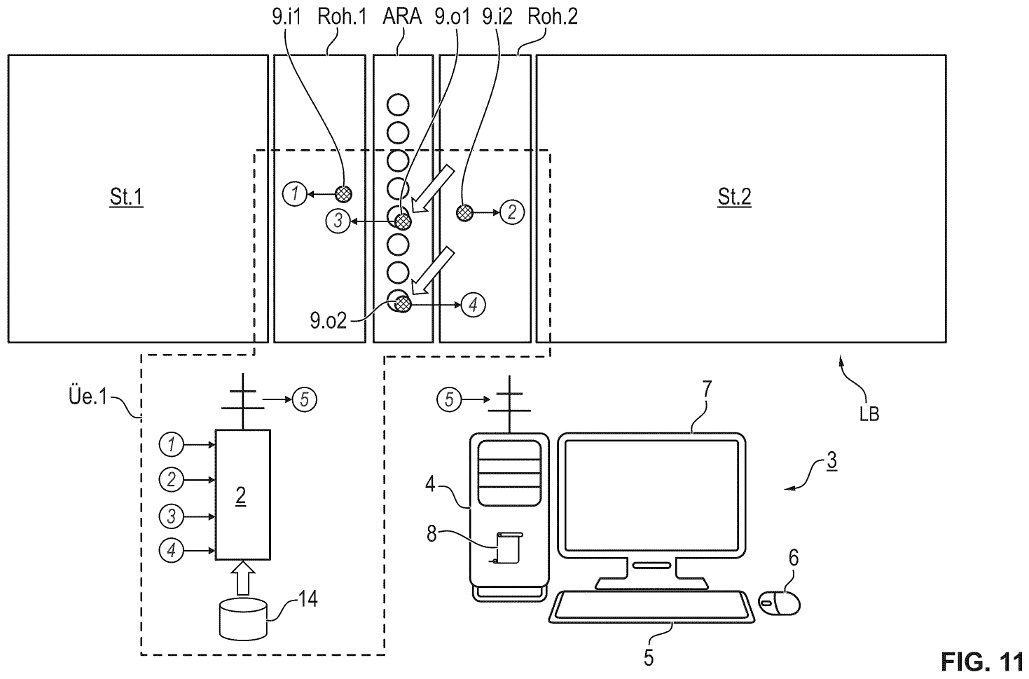

In the following, an embodiment is described with reference to FIG. 11. According to this embodiment, it is in particular possible to check at least one of the data connections of the monitoring assembly according to the invention. The subsequent description refers to the embodiment according to FIG. 1 in which two raw gas sensors 1.i1, 1.i2 and two clean gas sensors 1.o1, 1.o2 are used for monitoring the exhaust gas purification plant ARA. In a corresponding way, it is possible to check the monitoring assembly according to FIG. 2.

According to the embodiment described below, the monitoring assembly can selectively be operated in a monitoring mode or in a checking mode. In the monitoring mode every ammonia sensor 1.i1, 1.i2, 1.o1, 1.o2 generates and delivers a respective signal. This signal comprises information on the respective measured ammonia content. The evaluation program 8 determines, for the exhaust gas purification plant ARA and optionally for the or every further monitored exhaust gas purification plant ARA′, the respective ammonia content in the raw gas and the respective ammonia content in the clean gas. As described above, this determination is made for every sample time point.

In the following, it is described how the monitoring assembly is operated in the checking mode. In the checking mode, every ammonia sensor 1.i1, 1.i2, 1.o1, 1.o2 is replaced with a respective signal generator 9.i1, 9.i2, 9.o1, 9.o2. Preferably it is ensured in advance that every signal generator 9.i1, 9.i2, 9.o1, 9.o2 operates perfectly. In the following the terms raw gas signal generator 9.i1, 9.i2 and clean gas signal generator 9.o1, 9.o2 are used.

In one Implementation, every ammonia sensor 1.i1, 1.i2, 1.o1, 1.o2 is physically replaced with a respective signal generator 9.i1, 9.i2, 9.o1, 9.o2. In a further implementation the first monitoring unit Ue.1 permanently comprises every ammonia sensor as well as every signal generator 9.i1, 9.i2, 9.o1, 9.o2. While operated in the monitoring mode, the ammonia sensors 1.i1, 1.i2, 1.o1, 1.o2 are activated, and the signal generators 9.i1, 9.i2, 9.o1, 9.o2 are deactivated. In contrast, in the checking mode the ammonia sensors 1.i1, 1.i2, 1.o1, 1.o2 are deactivated, and the signal generators 9.i1, 9.i2, 9.o1, 9.o2 are activated. In one implementation, every signal generator 9.i1, 9.i2, 9.o1, 9.o2 is a part of a respective ammonia sensor 1.i1, 1.i2, 1.o1, 1.o2 and is an electronic circuit, e.g.

Preferably, every signal generator 9.i1, 9.i2, 9.o1, 9.o2 comprises its own energy supply unit. Every signal generator 9.i1, 9.i2, 9.o1, 9.o2 generates and delivers a respective signal which signal comprises an information on an ammonia content. In one implementation, this signal is an electrical signal, and a further implementation it is a digital signal. The signal generated by a signal generator 9.i1, 9.i2, 9.o1, 9.o2 has the same data format than the signal of an ammonia sensor 1.i1, 1.i2, 1.o1, 1.o2.

The ammonia content in the generated signal has not been measured. In contrast, the ammonia content was given by a user or by the evaluation program 8 or by a further part. The signal delivered by the signal generator 9.i1, 9.i2, 9.o1, 9.o2 comprises information of an ammonia content given by a user or by a device. Preferably, every signal generator 9.i1, 9.i2, 9.o1, 9.o2 comprises an input unit by which a user can give (enter) an ammonia content. It is also possible that every signal generator 9.i1, 9.i2, 9.o1, 9.o2 can be controlled remotely, for example by the evaluation program 8, and by a remote control a value for an ammonia content can be given to the signal generator. To summarize, every signal generator 9.i1, 9.i2, 9.o1, 9.o2 replaces and emulates a respective ammonia sensor 1.i1, 1.i2, 1.o1, 1.o2.

As explained above, the evaluation program 8 determines in the monitoring mode for every sample time point the respective ammonia content in the raw gas and the respective ammonia content in the clean gas. For this determination, the evaluation program 8 processes received signals of the ammonia sensors 1.i1, 1.i2, 1.o1, 1.o2. In addition, the evaluation program 8 computes for every sample time point a purification efficiency Rg=Rg(Ammᵣₑᵢₙ, Ammᵣₒₕ).

In the checking mode, a respective value for the ammonia content in the raw gas is given to the or every raw gas signal generator 9.i1, 9.i2. If several raw gas signal generators 9.i1, 9.i2 are used, in one implementation the same ammonia content value Ammᵣₒₕ is given to every raw gas signal generator 9.i1, 9.i2. In an alternative implementation, two different values for the ammonia content are given, for example Ammᵣₒₕ+Δ and Ammᵣₒₕ−Δ, resp. By this alternative implementation, it is possible to check whether or not the evaluation unit 9 aggregates measured values properly to a single value. The corresponding holds for the clean gas signal generators 9.o1, 9.o2.

The signals with the respective given values for the ammonia content are transmitted from the signal generators 9.i1, 9.i2, 9.o1, 9.o2 to the central computer 3 and are evaluated by the evaluation program 8. When operated in the checking mode, the evaluation program 8 also determines a respective ammonia content. For this determination, the evaluation program 8 processes the received signals of the signal generators 9.i1, 9.i2, 9.o1, 9.o2.

The evaluation program 8 determines a value for the ammonia content in the raw gas. For doing so, the evaluation program 8 processes the signals of the raw gas signal generators 9.i1, 9.i2. Correspondingly, the evaluation program 8 determines a value for the ammonia content in the clean gas. For doing so, the evaluation program 8 processes the signals of the clean gas signal generators 9.o1, 9.o2.

In one implementation, the evaluation program 8 “knows” for every signal generator 9.i1, 9.i2, 9.o1, 9.o2 which value for an ammonia content is given to this signal generator. The evaluation program 8 compares the determined values for the ammonia content with the respective given values. The determined values are received by processing the signals of the signal generators 9.i1, 9.i2, 9.o1, 9.o2. In a further implementation, a user performs this comparison.

According to both implementations, it is checked whether or not a determined value for an ammonia content differs from the respective given value about more than a given tolerance. In particular, possible reasons for a difference larger than a given tolerance are the following ones:
A part of the monitoring assembly is not at all or at least not sufficiently supplies with electrical energy. This applies for a part which has an own energy supply unit as well as for a part which is at least temporarily connected to a stationary energy supply network.
A data connection from a signal generator 9.i1, 9.i2, 9.o1, 9.o2 to the central computer 3 is interrupted or malfunctions in a further way.
A signal processing unit which is installed on the path from a signal generator to the evaluation program 8 delivers faulty values.

In an implementation of the embodiment with the signal generators, a respective temporal course of an ammonia content is given to every signal generator 9.i1, 9.i2, 9.o1, 9.o2. Preferably, by giving the temporal courses, the ammonia content is varied in such a way that at least once the value is 0 and at least once the maximal possible value for the ammonia content is taken. By this implementation, the applied signal processing for the entire value range of the ammonia content is checked. This holds for the raw gas as well as for the clean gas. In addition, the implementation that temporal courses are used facilitates to detect a relevant temporal delay when transmitting signals, compared with an implementation wherein only one respective value is given.

In particular, the evaluation program 8 is configured to automatically recognize if currently ammonia sensors 1.i1, 1.i2, 1.o1, 1.o2 or signal generators 9.i1, 9.i2, 9.o1, 9.o2 are used. With other words: The evaluation program 8 can automatically distinguish between an operation in the monitoring mode and an operation in the checking mode. In one implementation every signal generated by a signal generator 9.i1, 9.i2, 9.o1, 9.o2 comprises the given ammonia content and in addition a respective information, in particular an identifier of the signal generator 9.i1, 9.i2, 9.o1, 9.o2 or information that the ammonia content in the transmitted signal is given and has not been measured. It is also possible to give to every signal generator 9.i1, 9.i2, 9.o1, 9.o2 as the temporal course of the ammonia content a test pattern. This test pattern does not occur during a real operation of the monitored exhaust gas purification plant ARA.

### LIST OF REFERENCE SIGNS

1 Ammonia sensor
1.i1, 1.i2 Raw gas sensors of the first monitoring unit Ue.1, arranged upstream of the exhaust gas purification plant ARA
1′.i1, 1′.i2 Raw gas sensors of the further monitoring unit Ue.2, arranged upstream of the exhaust gas purification plant ARA′
1.o1, 1.o2 Clean gas sensors of the first monitoring unit Ue.1, arranged downstream of the exhaust gas purification plant ARA
1′.o1 Clean gas sensor of the further monitoring unit Ue.2, arranged downstream of the exhaust gas purification plant ARA′
2 Communication unit, receives signals from the raw gas sensors 1.i1, 1.i2 and from the clean gas sensors 1.o1, 1.o2, generates a respective unique sensor identifier for each signal and transmits the signals with the sensor identifiers to the central computer 3
3 Central computer 3, comprises the computing unit 4, the screen 7, the keyboard 5, and the mouse 6, receives signals from the communication unit 2
4 Computing unit of the central computer 3
5 Keyboard of the central computer 3
6 Mouse of the central computer 3
7 Screen of the central computer 3
8 Evaluation program on the central computer 3, evaluates the signals from the sensors 1.i1, 1.i2, 1.o1, 1.o2
8.1 Further evaluation program
9.i1, 9.i2 Raw gas signal generator which replaces the raw gas sensors 1.i1, 1.i2 during an operation in the checking mode
9.o1, 9.o2 Clean gas signal generator which replaces the clean gas sensors 1.o1, 1.o2 during an operation in the checking mode
10 Feed unit, comprises the inlet opening 22 and the outlet opening 21
11 Eyelet on the housing 24
12 Cable on the housing 24, allows ammonia sensor 1 to be electrically connected to a stationary power supply network
14 Data memory in which the unique identifiers of the sensors 1.i1, 1.i2, 1.o1, 1.o2 and the unique identifier of the monitoring unit Ue.1, Ue.2 are stored and to which the communication unit 2 has at least temporary read access
20 Wall of the feed unit 10
21 Outlet opening from the feed unit 10
22 Inlet opening into the feed unit 10
23 Region in the feed unit 10 in which the gas sample settles
24 Housing, surrounds the interior region 140 with the power supply unit, carries the eyelet 11 and the retaining cable 12
27 Inlet gap between the feed unit 10 and the impact protection
30 Heating element in the wall 20
40 Mechanical impact protection below the feed unit 10
50 Measuring electrode
51 Counter electrode
52 Reference electrode
53 Electrolyte
54 Potentiostat
55 Display unit
56 Porous membrane
57 Housing
100 Sensor cell with the measuring chamber, measures the ammonia content
110 Region of the measuring chamber which receives a gas sample flowing in from below, is delimited by the wall 130, the outlet opening 21, and the membrane 121 and belongs to the sensor cell 100
120 Protective filter in front of the sensor cell 100
121 Membrane that separates the region 110 for a gas sample from an electrolyte in the measuring chamber
130 Wall of the measuring chamber of the sensor cell 100
140 Interior region of the housing 24, accommodates a power supply unit and a signal-processing evaluation unit
Ammᵣₑᵢₙ Ammonia content in the clean gas mixture, measured by the clean gas sensors 1.o1, 1.o2
Ammᵣₒₕ Ammonia content in the raw gas mixture, measured by the raw gas sensors 1.i1, 1.i2
ARA, ARA′ Exhaust gas purification plant, removes ammonia from raw gas and thereby provides the clean gas mixture, is monitored by the monitoring unit Ue.1, Ue.2
Auf Collection container, which collects the washing water with the ammonium sulfate, belongs to the exhaust gas purification plant ARA
Δ Difference between the ammonia content Ammᵣₒₕ in the raw gas and the ammonia content Ammᵣₑᵢₙ in the clean gas mixture
dur(F_Zr) Duration of the malfunction period F_Zr
F_Zr, F_Zr1, . . . Malfunction period, i.e., a period in which the purification efficiency Rg is continuously smaller than a specified lower limit
Gb Building which houses the stable St, the technical room Tr and the exhaust gas purification plant WW, Auf
Gg Gas mixture in which the ammonia content is measured
K Number of different operating principles of an exhaust gas purification plant that the evaluation program 8 is able to evaluate
LB, LB′ Agricultural enterprise with the exhaust gas purification plant ARA, ARA′
LW Conductivity sensor that measures the electrical conductivity of the washing water in the collection container Auf
M Maximum possible number of raw gas sensors that the evaluation program 8 is able to evaluate
N Maximum possible number of clean gas sensors that the evaluation program 8 is able to evaluate
pH pH sensor that measures the pH value of the washing water in the collection container Auf
Rg Purification efficiency of the exhaust gas purification plant ARA, depends on Ammᵣₒₕ and Ammᵣₑᵢₙ
St, St.1, St.2 Stable for animal breeding
Roh.1, Roh.2 Region, in which raw gas can accumulate, between the stable St.1, St.2 and the exhaust gas purification plant ARA
StE Aeration for the stable St, comprises the first fan Vent1
StB Deaeration for the stable St
Tr Technical room
Ue.1 First monitoring unit, comprises the raw gas sensors 1.i1, 1.i2, the clean gas sensors 1.o1, 1.o2 and the communication unit 2, monitors the exhaust gas purification plant ARA
Ue.2 Second (further) monitoring unit, comprises the raw gas sensors 1′.i1, 1′.i2, the clean gas sensor 1′.o1 and the communication unit 2′, monitors the exhaust gas purification plant ARA′
U_Zr, U_Zr.1, . . . Monitoring period
Vb Swirl region in which the raw gas sensor 1.i1 is arranged
Vent1 First fan, arranged in the aeration region StE, extracts raw gas from the stable St
Vent2 Second fan, arranged on the roof of the building Gb, extracts the clean gas mixture from the building Gb
Vr.i Availability rate of the exhaust gas purification plant ARA in the monitoring period U_Zr.i
Vr.avg Average availability rate of the exhaust gas purification plant ARA
WW Scrubber Wall of the Chemical Exhaust Gas Purification Plant ARA, adds sulfuric acid to the raw gas, belongs to the exhaust gas purification plant ARA

1.-18. (canceled)

19. A monitoring assembly for monitoring an exhaust gas purification plant:
wherein the monitored exhaust gas purification plant is configured to:
reduce the content of ammonia in a raw gas, and
provide, by reducing the content of ammonia, a clean gas with a reduced ammonia content,
the monitoring assembly comprising:
a monitoring unit; and
a signal-processing evaluation unit,
wherein the monitoring unit comprises:
a raw gas sensor; and
a clean gas sensor,
wherein the raw gas sensor of the monitoring unit is configured to:
measure the ammonia content in the raw gas; and
generate a raw gas signal,
wherein the raw gas signal comprises information about the ammonia content in the raw gas, the ammonia content being measured by the raw gas sensor,
wherein the clean gas sensor of the monitoring unit is configured to:
measure the ammonia content in the clean gas; and
generate a clean gas signal,
wherein the clean gas signal comprises information about the ammonia content in the clean gas, the ammonia content being measured by the clean gas sensor,
wherein the monitoring assembly is configured to transmit the raw gas signal and the clean gas signal to the evaluation unit,
wherein a monitoring period, a quality function, and a lower quality limit are specified,
wherein the quality function is based on:
the ammonia content in the clean gas; and
the ammonia content in the raw gas,
such that a functional value of the quality function increases as the ammonia content in the clean gas decreases provided that the ammonia content in the raw gas is a constant value,
wherein the evaluation unit is configured to:
determine, based on the raw gas signal and the clean gas signal, the ammonia content in the raw gas and the ammonia content in the clean gas; and
determine, based on the determined ammonia content in the raw gas and on the determined ammonia content in the clean gas, one or more malfunction periods occurring in the specified monitoring period, or
determine that there is no malfunction period in the specified monitoring period,
wherein a given malfunction period is a time period in which each functional value of the specified quality function is below the lower quality limit.

20. The monitoring assembly of claim 19:
wherein the raw gas sensor is a first raw gas sensor and the monitoring unit comprises a second raw gas sensor,
wherein the raw gas signal is a first raw gas signal and the second raw gas sensor is configured to:
measure the ammonia content in the raw gas; and
generate a second raw gas signal,
wherein the evaluation unit is configured to determine whether a specified raw gas failure criterion is met,
wherein the raw gas failure criterion is fulfilled if at least one of a criterion of a group comprising the following criteria is fulfilled:
either the absolute deviation or the percentage deviation between the ammonia content indicated by the first raw gas signal and the ammonia content indicated by the second raw gas signal exceeds a specified lower limit,
a change in either the absolute deviation or the percentage deviation between the ammonia content indicated by the first raw gas signal and the ammonia content indicated by the second raw gas signal exceeds a specified lower limit, and
wherein the evaluation unit is further configured to determine:
if the specified failure criterion is not met, the ammonia content in the raw gas based on the first raw gas and based on the second raw gas signals, and,
if the specified failure criterion is met, the ammonia content in the raw gas depending only on the first raw gas signal if the ammonia content indicated in the first raw gas signal exceeds the ammonia content indicated in the second raw gas signal and depending only on the second raw gas signal if the ammonia content indicated in the second raw gas signal exceeds the ammonia content indicated in the first raw gas signal.

21. The monitoring assembly of claim 19:
wherein the clean gas sensor is a first clean gas sensor and the first monitoring unit comprises a second clean gas sensor;
wherein the clean gas signal is a first clean gas signal and the second clean gas sensor is configured to:
measure the ammonia content in the clean gas; and
generate a second clean gas signal,
wherein the evaluation unit is configured to determine whether a specified clean gas failure criterion is met,
wherein the clean gas failure criterion is fulfilled if at least one of a criterion of a group comprising the following criteria is fulfilled:
either of the absolute deviation or the percentage deviation between the ammonia content indicated by the first clean gas signal and the ammonia content indicated by the second clean gas signal exceeds a specified lower limit, or
a change in either of the absolute deviation or the percentage deviation between the ammonia content indicated by the first clean gas sensor and the ammonia content indicated by the second clean gas signal exceeds a specified lower limit, and
wherein the evaluation unit is further configured to determine:
if the specified failure criterion is not met, the ammonia content in the clean gas based on the first and on the second clean gas signal, and,
if the specified failure criterion is met, the ammonia content in the clean gas depending only on the first clean gas signal if the ammonia content indicated in the first clean gas signal exceeds the ammonia content indicated in the second clean gas signal and depending only on the second clean gas signal if the ammonia content indicated in the second clean gas signal exceeds the ammonia content indicated in the first clean gas signal.

22. The monitoring assembly according to claim 19:
wherein the evaluation unit is configured to:
determine, for the specified monitoring period, a time span in the monitoring period during which, for the exhaust gas purification plant, one or more functional values of the specified quality function were below the specified lower limit; and
determine, depending on the determination of the time span, an availability rate of the exhaust gas purification plant during the monitoring period,
wherein the availability rate indicates an entire time proportion, in the monitoring period, of periods in which the one or more functional values of the quality function met or exceeded the specified lower limit.

23. The monitoring assembly of claim 22:
wherein a sequence comprising at least two non-overlapping monitoring periods is specified, and
wherein the evaluation unit is configured to:
generate a graphical representation; and
cause the generated graphical representation to be output in at least one form that can be perceived by a human,
wherein the graphical representation shows, for each specified monitoring period of the sequence, a respective availability rate that the exhaust gas purification plant has achieved in the respective specified monitoring period.

24. The monitoring assembly of claim 19:
wherein the evaluation unit is configured to:
generate a graphical representation with a first axis for time and a second axis for a determined ammonia content; and
cause the generated representation to be output in at least one form that can be perceived by a human,
wherein the generated graphical representation shows a temporal course of the determined ammonia content in the clean gas over the monitoring period and shows the one or more malfunction periods, and
wherein, for each malfunction period of the one or more malfunction periods, a section of the temporal course that falls within this given malfunction period is represented in a first manner, and
wherein each section of the temporal course that does not fall within any malfunction period of the one or more malfunction periods is represented in a second manner that differs from the first manner.

25. The monitoring assembly according to claim 19:
wherein a gas sensor of the monitoring unit comprises a sensor cell, wherein the gas sensor is the raw gas sensor or the clean gas sensor,
wherein the sensor cell comprises:
a measuring chamber;
a tubular feed unit; and
a heating element,
wherein, while the monitoring assembly is used, the feed unit is arranged vertically or obliquely below the measuring chamber,
wherein the heating element is configured to:
heat the interior of the feed unit; and
cause, based on heating the interior of the feed unit, a convection flow to be generated in the feed unit,
wherein the generated convection flow conveys a gas sample from the environment through the feed unit vertically or obliquely upward into the measuring chamber, and
wherein the sensor cell is configured to measure the ammonia content in the gas sample conveyed into the measuring chamber.

26. The monitoring assembly of claim 19:
wherein the monitoring unit comprises, for a gas sensor of the monitoring unit, a tubular protective element, wherein the gas sensor is the raw gas sensor or the clean gas sensor,
wherein the tubular protective element extends along a longitudinal axis,
wherein, while the monitoring assembly is used, the longitudinal axis of the protective element is arranged vertically or obliquely, and
wherein the gas sensor is arranged inside the protective element.

27. The monitoring assembly of claim 19:
wherein the monitoring unit further comprises a communication unit,
wherein the monitoring assembly comprises a central computer, wherein the central computer is arranged at a distance from the monitoring unit,
wherein the monitoring unit is configured to establish, at least temporarily, a first data connection between the raw gas sensor and the communication unit and a second data connection between the clean gas sensor and the communication unit,
wherein the monitoring assembly is further configured to establish, at least temporarily, an additional data connection between the communication unit and the central computer,
wherein the communication unit is configured to
receive from the raw gas sensor the raw gas signal and from the clean gas sensor the clean gas signal;
generate a raw gas message comprising the raw gas signal and a clean gas message comprising the clean gas signal; and
cause the raw gas message and the clean gas message to be transmitted to the central computer, and
wherein the evaluation unit is at least one of the following:
a component of the central computer, or
executable on the central computer.

28. The monitoring assembly of claim 27:
wherein the monitoring unit is a first monitoring unit, the communication unit is a first communication unit, and the monitoring assembly comprises a further monitoring unit,
wherein the central computer is arranged at a distance from the further monitoring unit,
wherein the further monitoring unit comprises:
a further raw gas sensor;
a further clean gas sensor; and
a further communication unit,
wherein the further monitoring unit is configured to establish, at least temporarily, a third data connection between the further raw gas sensor and the further communication unit and a fourth data connection between the further clean gas sensor and the further communication unit,
wherein the monitoring assembly is configured to establish, at least temporarily, a further additional data connection between the further communication unit and the central computer,
wherein the exhaust gas purification plant to which the first monitoring unit is assigned is a first exhaust gas purification plant, and
wherein the further monitoring unit is assigned to a further exhaust gas purification plant.

29. The monitoring assembly of claim 27:
wherein the communication unit comprises a data memory,
wherein a communication unit identifier for the first communication unit, a raw gas sensor identifier for the raw gas sensor, and a clean gas sensor identifier for the clean gas sensor are stored on the data memory,
wherein the communication unit identifier distinguishes the first communication unit from one or more other communication units corresponding to the central computer,
wherein the raw gas sensor identifier distinguishes the raw gas sensor from one or more other sensors of the monitoring unit,
wherein the clean gas sensor identifier distinguishes the clean gas sensor from one or more other sensors of the monitoring unit, and
wherein the communication unit is configured to:
generate the raw gas message such that it additionally comprises the communication unit identifier and the raw gas sensor identifier; and
generate the clean gas message such that it additionally comprises the communication unit identifier and the clean gas sensor identifier.

30. The monitoring assembly of claim 29:
wherein a number identifier is stored on the data memory, the number identifier indicating an amount of raw gas sensors and an amount of clean gas sensors that the first monitoring unit comprises,
wherein the communication unit is configured to:
generate the raw gas message such that it additionally comprises the number identifier; and
generate the clean gas message such that it additionally comprises the number identifier.

31. The monitoring assembly of claim 19:
wherein the monitoring unit additionally comprises at least one of:
a conductivity sensor, or
a pH sensor,
wherein the conductivity sensor is configured to:
measure the electrical conductivity of a cleaning liquid used to reduce the ammonia content in the raw gas; and
generate a conductivity signal comprising the measured electrical conductivity,
wherein the pH sensor is configured to:
measure a pH value of the cleaning liquid; and
generate a pH value signal comprising the measured pH value,
wherein the evaluation unit is configured to:
determine one or more periods, in the monitoring period, in which at least one of the following conditions occurs:
the measured pH value is below the specified lower limit, or
the measured electrical conductivity exceeds a specified upper limit; and
use each determined period as a malfunction period.

32. The monitoring assembly of claim 31:
wherein the communication unit comprises a data memory,
wherein an operating identifier indicating whether the exhaust gas purification plant monitored by the monitoring unit works biologically or chemically is stored in the data memory, and
wherein the evaluation unit is configured to compare the measured electrical conductivity and the measured pH value with a respective limit only if the monitored exhaust gas purification plant works biologically.

33. The monitoring assembly of claim 19:
wherein the monitoring assembly comprises, for each one of the raw gas sensor and the clean gas sensor, a respective signal generator,
wherein the respective signal generator is configured to:
capture a respective given value of an ammonia content; and
generate a generator signal such that the generator signal comprises information about the ammonia content value captured by the signal generator,
wherein the monitoring assembly is configured to selectively be operated in a monitoring mode or in a checking mode,
wherein the monitoring assembly being operated in the monitoring mode is configured to use the raw gas sensor and the clean gas sensor of the first monitoring unit,
wherein the monitoring assembly being operated in the checking mode is configured to:
use, instead of the raw gas sensor and the clean gas sensor, the respective signal generator; and
transmit the respective generator signal to the evaluation unit, and
wherein with the monitoring assembly being operated in the checking mode, the evaluation unit is configured to:
determine, for each respective signal generator, an ammonia content value which is given to the signal generator; and
use the transmitted generator signal of the signal generator for determining the ammonia content value.

34. A system comprising:
an exhaust gas purification plant; and
a monitoring assembly according to claim 19,
wherein the gas purification plant is configured to:
reduce the content of ammonia in a raw gas and
provide, based on reducing the content of ammonia, a clean gas with a reduced ammonia content compared to the raw gas, and
wherein the monitoring unit of the monitoring assembly is assigned to the exhaust gas purification plant, and
wherein the monitoring assembly is configured to monitor the exhaust gas purification plant of the system.

35. The system of claim 34:
wherein the system further comprises a building,
wherein the exhaust gas purification plant is located in the building or is located adjacent to the building, and
wherein the building comprises a stable for keeping animals.

36. A monitoring method for monitoring an exhaust gas purification plant:
wherein the monitored exhaust gas purification plant is configured to:
reduce the content of ammonia in a raw gas; and
provide, by reducing the content of ammonia, a clean gas with a reduced ammonia content,
wherein the monitoring method is carried out using a monitoring assembly, the monitoring assembly comprising:
a monitoring unit; and
a signal-processing evaluation unit,
wherein the monitoring unit comprises:
a raw gas sensor; and
a clean gas sensor,
wherein a monitoring period, a quality function, and a lower quality limit are specified,
wherein the quality function depends on:
the ammonia content in the clean gas; and
the ammonia content in the raw gas,
such that a functional value of the quality function increases as the ammonia content in the clean gas decreases provided a constant ammonia content in the raw gas,
wherein the method comprises the automatically performed steps:
with the raw gas sensor, repeatedly measuring the ammonia content in the raw gas;
generating a raw gas signal, wherein the generated raw gas signal comprises information about the measured ammonia content in the raw gas;
with the clean gas sensor, repeatedly measuring the ammonia content in the clean gas;
generating a clean gas signal, wherein the generated clean gas signal comprises information about the measured ammonia content in the clean gas;
transmitting the raw gas signal and the clean gas signal to the evaluation unit;
with the evaluation unit, based on the raw gas signal and the clean gas signal, determining:
the ammonia content in the raw gas; and
the ammonia content in the clean gas; and
with the evaluation unit, based on the determined ammonia content in the raw gas and the determined ammonia content in the clean gas, determining one or more malfunction periods occurring in the specified monitoring period, or
determining that there is no malfunction period in the specified monitoring period,
wherein a given malfunction period is a period in which each functional value of the specified quality function is below the lower quality limit.

37. The monitoring method of claim 36:
wherein the monitoring unit additionally comprises a communication unit,
wherein the monitoring assembly comprises a central computer arranged at a distance from the first monitoring unit,
wherein the evaluation unit is at least one of:
a component of the central computer, or
executed on the central computer, and
wherein the method comprises the additional steps of:
at least temporarily establishing a data connection between the raw gas sensor and the communication unit and a data connection between the clean gas sensor and the communication unit;
at least temporarily establishing an additional data connection between the communication unit and the central computer; and
with the communication unit:
receiving from the raw gas sensor the raw gas signal and receiving from the clean gas sensor the clean gas signal;
generating a raw gas message comprising the raw gas signal and a clean gas message comprising the clean gas signal; and
causing the raw gas message and the clean gas message being transmitted to the central computer.

38. The monitoring method of claim 36:
wherein the monitoring assembly comprises, for each one of the raw gas sensor and the clean gas sensor, a respective signal generator,
wherein the monitoring assembly is checked at least once by a checking method,
wherein the checking method comprises the steps:
instead of using the raw gas sensor and the clean gas sensor, using the respective signal generator;
capturing, with each signal generator, a respective given ammonia content value; and
with each signal generator, generating a respective generator signal,
wherein the generated generator signal comprises information about the ammonia content value given to the respective signal generator,
wherein the generator signals are transmitted to the evaluation unit, and
wherein based on the generator signals, the evaluation unit determines every ammonia content value given to the respective signal generator.
